SEQUENCE LISTING (95 sequences)

SEQ ID NO: 1 - protein, 36 aa, Leiurus quinquestriatus
  misc_feature: Chlorotoxin
MCMPCFTTDHQMARKCDDCCGGKGRGKCYGPQCLCR

SEQ ID NO: 2 - protein, 42 aa, Leiurus quinquestriatus
HHHHHHMCMPCFTTDHQMARKCDDCCGGKGRGKCYGPQCLCR

SEQ ID NO: 3 - protein, 37 aa, Leiurus quinquestriatus
YMCMPCFTTDHQMARKCDDCCGGKGRGKCYGPQCLCR

SEQ ID NO: 4 - protein, 39 aa, Leiurus quinquestriatus
YSYMCMPCFTTDHQMARKCDDCCGGKGRGKCYGPQCLCR

SEQ ID NO: 5 - protein, 36 aa, Artificial sequence
  Chlorotoxin variant
MCMPCFTTDHQMARKCDDCCGGKGRGKCFGPQCLCR

SEQ ID NO: 6 - protein, 35 aa, Artificial sequence
  Chlorotoxin variant
RCKPCFTTDPQMSKKCADCCGGKGKGKCYGPQCLC

SEQ ID NO: 7 - protein, 38 aa, Artificial sequence
  Chlorotoxin variant
RCSPCFTTDQQMTKKCYDCCGGKGKGKCYGPQCICAPY

SEQ ID NO: 8 - protein, 7 aa, Leiurus quinquestriatus
  misc_feature: Derivative of Chlorotoxin  amino acid residues 23-29
KGRGKSY

SEQ ID NO: 9 - protein, 7 aa, Leiurus quinquestriatus
  misc_feature: Derivative of Chlorotoxin  amino acid residues 8-14
TDHQMAR

SEQ ID NO: 10 - protein, 9 aa, Artificial sequence
  Chlorotoxin alpha peptide
TDHQMARKS

SEQ ID NO: 11 - protein, 9 aa, Artificial sequence
  Variant of chlorotoxin alpha peptide
TAHAMARKS

SEQ ID NO: 12 - protein, 36 aa, Artificial sequence
  Variant peptide of chlorotoxin
MCMPCFTTAHAMARKCDDCCGGKGRCKCYGPQCLCR

SEQ ID NO: 13 - protein, 9 aa, Artificial
  motif for chlorotoxin derivatives
TTXXXMXXK

SEQ ID NO: 14 - protein, 9 aa, Leiurus quinquestriatus
TTDHQMARK

SEQ ID NO: 15 - protein, 35 aa, Mesobuthus tamulus; identical to SEQ ID NO: 6

SEQ ID NO: 16 - protein, 34 aa, Artificial sequence
  Small Toxin consensus sequence
CXPCFTTDXQMAKKCXDCCGGKGKGKCYGPQCLC

SEQ ID NO: 17 - protein, 38 aa, Leiurus quinquestriatus; identical to SEQ ID NO: 7

SEQ ID NO: 18 - protein, 34 aa, Artificial sequence
  Probable Toxin LQH 8/6 consensus sequence
CXPCFTTDXQMXKKCXDCCGGKGKGKCYGPQCIC

SEQ ID NO: 19 - protein, 61 aa, Mesobuthus martensii
  MISC_FEATURE (1)..(61): Xaa can be any amino acid
MKFLYGIVFIALFLTVMFATQTDGCGPCFTTDANMARKCRECCGGIGXXKCFGPQCLCNRI

SEQ ID NO: 20 - protein, 34 aa, Artificial sequence
  Chinese Scorpion consensus sequence
CXPCFTTDXNMARKCXDCCGGXGXXKCFGPQCLC

SEQ ID NO: 21 - protein, 59 aa, Mesobuthus martensii
MKFLYGIVFIALFLTVMFATQTDGCGPCFTTDANMARKCRECCGGIGKCFGPQCLCNRI

SEQ ID NO: 22 - protein, 32 aa, Artificial sequence
  Chinese Scorpion consensus sequence
CXPCFTTDXNMARKCXDCCGGXGKXXXXXXXC

SEQ ID NO: 23 - protein, 37 aa, Mesobuthus eupeus
  MISC_FEATURE (1)..(37): Xaa can be any amino acid
MCMPCFTTDPNMANKCRDCCGGXGKXKCFGPQCLCNR

SEQ ID NO: 24 - protein, 35 aa, Artificial sequence
  Insect toxin I5 consensus sequence
MCMPCFTTDXNMAXKCXDCCGGXGKXKCFGPQCLC

SEQ ID NO: 25 - protein, 35 aa, Mesobuthus eupeus
MCMPCFTTDPNMANKCRDCCGGGKKCFGPQCLCNR

SEQ ID NO: 26 - protein, 33 aa, Artificial sequence
  Insect toxin I5 consensus sequence
MCMPCFTTDXNMAXKCXDCCGGXXKXXXXXXXC

SEQ ID NO: 27 - protein, 38 aa, Mesobuthus eupeus
  MISC_FEATURE (1)..(38): Xaa can be any amino acid
MCMPCFTTRPDMAQQCRACCKGXXRGKCFGPQCLCGYD

SEQ ID NO: 28 - protein, 35 aa, Artificial sequence
  Insectotoxin I1 consensus sequence
MCMPCFTTXXXMAXXCXXCCXGXXRGKCFGPQCLC

SEQ ID NO: 29 - protein, 36 aa, Mesobuthus eupeus
MCMPCFTTRPDMAQQCRACCKGRGKCFGPQCLCGYD

SEQ ID NO: 30 - protein, 33 aa, Artificial sequence
  Insectotoxin I1 consensus sequence
MCMPCFTTXXXMAXXCXXCCXGKGKXXXXXXXC

SEQ ID NO: 31 - protein, 37 aa, Mesobuthus eupeus
  MISC_FEATURE (1)..(37): Xaa can be any amino acid
MCMPCFTTDPNMAKKCRDCCGGNGXXKCFGPQCLCNR

SEQ ID NO: 32 - protein, 35 aa, Artificial sequence
  Insectotoxin 15A consensus sequence
MCMPCFTTDXNMAKKCXDCCGGXGXXKCFGPQCLC

SEQ ID NO: 33 - protein, 35 aa, Mesobuthus eupeus
MCMPCFTTDPNMAKKCRDCCGGNGKCFGPQCLCNR

SEQ ID NO: 34 - protein, 33 aa, Artificial sequence
  Insectotoxin 15A consensus sequence
MCMPCFTTDXNMAKKCXDCCGGXGKXXXXXXXC

SEQ ID NO: 35 - protein, 37 aa, Androctonus mauretanicus
  MISC_FEATURE (1)..(37): Xaa can be any amino acid
CGPCFTTDPYTESKCATCCGGXXRGKCVGPQCLCNRI

SEQ ID NO: 36 - protein, 34 aa, Artificial sequence
  Neurotoxin P2 consensus sequence
CXPCFTTDXXXXXKCXXCCGGXXRGKCXGPQCLC

SEQ ID NO: 37 - protein, 35 aa, Androctonus mauretanicus
CGPCFTTDPYTESKCATCCGGRGKCVGPQCLCNRI

SEQ ID NO: 38 - protein, 32 aa, Artificial sequence
  Neurotoxin P2 consensus sequence
CXPCFTTDXXXXXKCXXCCGGKGKXXXXXXXC

SEQ ID NO: 39 - protein, 37 aa, Artificial sequence
  Toxin consensus sequence
MCMPCFTTDPNMAKKCRDCCGGKGXXKCFGPQCLCNR

SEQ ID NO: 40 - protein, 35 aa, Artificial sequence
  Toxin consensus sequence
RCXPCFTTDXQMSKKCXDCCGGKGKGKCYGPQCLC

SEQ ID NO: 41 - protein, 35 aa, Artificial sequence
  Toxin consensus sequence
MCMPCFTTDPNMARKCRDCCGGRGKCFGPQCLCNR

SEQ ID NO: 42 - protein, 10 aa, Artificial sequence
  Pep8-Ctlx
CGGKGRGKCY

SEQ ID NO: 43 - protein, 10 aa, Artificial sequence
  Pep8-SCX1_BUTSI
CGGKGKGKCY

SEQ ID NO: 44 - protein, 10 aa, Artificial sequence
  Pep8-AF079059_2
CGGIGKCFGP

SEQ ID NO: 45 - protein, 12 aa, Artificial sequence
  Chlorotoxin Peptide 8 consensus sequence
CGGXGRGKCFGP

SEQ ID NO: 46 - protein, 6 aa, Artificial sequence
  Chlorotoxin Peptide 8 consensus sequence
CGGXGK

SEQ ID NO: 47 - protein, 10 aa, Artificial sequence
  Pep8-NJ0361 sequence
CGGGKKCFGP

SEQ ID NO: 48 - protein, 12 aa, Artificial sequence
  Chlorotoxin Peptide 8 consensus sequence
CGGKGKGKCFGP

SEQ ID NO: 49 - protein, 6 aa, Artificial sequence
  Chlorotoxin Peptide 8 consensus sequence
CGGXXK

SEQ ID NO: 50 - protein, 10 aa, Artificial sequence
  Pep8-SCX1_BUTEU sequence
CKGRGKCFGP

SEQ ID NO: 51 - protein, 12 aa, Artificial sequence
  Chlorotoxin Peptide 8 consensus sequence
CGXKGRGKCFGP

SEQ ID NO: 52 - protein, 6 aa, Artificial sequence
  Chlorotoxin Peptide 8 consensus sequence
CXGKGK

SEQ ID NO: 53 - protein, 10 aa, Artificial sequence
  Pep8-SCX5_BUTEU sequence
CGGNGKCFGP

SEQ ID NO: 54 - protein, 12 aa, Artificial sequence
  Chlorotoxin Peptide 8 consensus sequence
CGGXGRGKCFGP

SEQ ID NO: 55 - protein, 6 aa, Artificial sequence
  Chlorotoxin Peptide 8 consensus sequence
CGGXGK

SEQ ID NO: 56 - protein, 10 aa, Artificial sequence
  Pep8-SCXP_ANDMA sequence
CGGRGKCVGP

SEQ ID NO: 57 - protein, 12 aa, Artificial sequence
  Chlorotoxin Peptide 8 consensus sequence
CGGKGRGKCXGP

SEQ ID NO: 58 - protein, 6 aa, Artificial sequence
  Chlorotoxin Peptide 8 consensus sequence
CGGKGK

SEQ ID NO: 59 - protein, 12 aa, Artificial sequence
  Chlorotoxin Peptide 8 consensus sequence
CGGXXRGKCFGP

SEQ ID NO: 60 - protein, 10 aa, Artificial sequence
  Chlorotoxin Peptide 8 consensus sequence
CGGKGKCFGP

SEQ ID NO: 61 - protein, 10 aa, Artificial sequence
  Chlorotoxin Peptide 21 sequence
TTDHQMARKC

SEQ ID NO: 62 - protein, 10 aa, Artificial sequence
  Pep21-SCX1-BUTSI sequence
TTDPQMSKKC

SEQ ID NO: 63 - protein, 10 aa, Artificial sequence
  Chlorotoxin Peptide 21 consensus sequence
TTDXQMAKKC

SEQ ID NO: 64 - protein, 10 aa, Artificial sequence
  Pep21-SCX8_LEIQH sequence
TTDQQMTKKC

SEQ ID NO: 65 - protein, 10 aa, Artificial sequence
  Chlorotoxin Peptide 21 consensus sequence
TTDXQMXKKC

SEQ ID NO: 66 - protein, 10 aa, Artificial sequence
  Pep21-AF079059_2 sequence
TTDANMARKC

SEQ ID NO: 67 - protein, 10 aa, Artificial sequence
  Chlorotoxin Peptide 21 consensus sequence
TTDXNMARKC

SEQ ID NO: 68 - protein, 10 aa, Artificial sequence
  Pep21-JN0361 sequence
TTDPNMANKC

SEQ ID NO: 69 - protein, 10 aa, Artificial sequence
  Chlorotoxin Peptide 21 consensus sequence
TTDXNMAXKC

SEQ ID NO: 70 - protein, 10 aa, Artificial sequence
  Pep21-SCX1_BUTEU sequence
TTRPDMAQQC

SEQ ID NO: 71 - protein, 10 aa, Artificial sequence
  Chlorotoxin Peptide 21 consensus sequence
TTXXXMAXXC

SEQ ID NO: 72 - protein, 10 aa, Artificial sequence
  Pep21-SCX5_BUTEU sequence
TTDPNMAKKC

SEQ ID NO: 73 - protein, 10 aa, Artificial sequence
  Chlorotoxin Peptide 21 consensus sequence
TTDXNMAKKC

SEQ ID NO: 74 - protein, 10 aa, Artificial sequence
  Pep21-SCXP_ANDMA sequence
TTDPYTESKC

SEQ ID NO: 75 - protein, 10 aa, Artificial sequence
  Chlorotoxin Peptide 21 consensus sequence
TTDXXXXXKC

SEQ ID NO: 76 - protein, 10 aa, Artificial sequence
  Chlorotoxin Peptide 21 consensus sequence
TTDPNMAKKC

SEQ ID NO: 77 - protein, 7 aa, Artificial sequence
  Chlorotoxin derivative STP-1
TDPQMSR

SEQ ID NO: 78 - protein, 10 aa, Artificial sequence
  Peptide 8 sequences
GGKGRGKSYG

SEQ ID NO: 79 - protein, 9 aa, Artificial sequence
  Peptide 8a sequence
GKGRGKSYG

SEQ ID NO: 80 - protein, 8 aa, Artificial sequence
  Peptide 8b sequence
KGRGKSYG

SEQ ID NO: 81 - protein, 7 aa, Artificial sequence
  Peptide 8c sequence
GRGKSYG

SEQ ID NO: 82 - protein, 10 aa, Artificial sequence
  Peptide 21 sequence
TTDHQMARKS

SEQ ID NO: 83 - protein, 8 aa, Artificial sequence
  Peptide 21b sequence
DHQMARKS

SEQ ID NO: 84 - protein, 7 aa, Artificial sequence
  Peptide 21c sequence
HQMARKS

SEQ ID NO: 85 - protein, 6 aa, Artificial sequence
  Peptide 21d sequence
QMARKS

SEQ ID NO: 86 - protein, 9 aa, Artificial sequence
  Peptide 21a-A1 sequence
ADHQMARKS

SEQ ID NO: 87 - protein, 9 aa, Artificial sequence
  Peptide 21a-A2 sequence
TAHQMARKS

SEQ ID NO: 88 - protein, 9 aa, Artificial sequence
  Peptide 21a-A3 sequence
TDAQMARKS

SEQ ID NO: 89 - protein, 9 aa, Artificial sequence
  Peptide 21a-A4 sequence
TDHAMARKS

SEQ ID NO: 90 - protein, 9 aa, Artificial sequence
  Peptide 21a-A5 sequence
TDHQAARKS

SEQ ID NO: 91 - protein, 9 aa, Artificial sequence
  Peptide 21a-A7 sequence
TDHQMAAKS

SEQ ID NO: 92 - protein, 9 aa, Artificial sequence
  Peptide 21a-A8 sequence
TDHQMARAS

SEQ ID NO: 93 - protein, 9 aa, Artificial sequence
  Peptide 21a-A9 sequence
TDHQMARKA

SEQ ID NO: 94 - protein, 9 aa, Mesobuthus tamulus sindicus
  GenBank Accesssion No. P15229, small toxin
TTDQQMSKK

SEQ ID NO: 95 - protein, 9 aa, Leiurus quinquestriatus hebraeu
  GenBank Accession No. P55966, probable toxin
TTDPQMSKK

(19) United States
(12) Patent Application Publication
(10) Pub. No.: US 2006/0166892 A1
(43) Pub. Date: Jul. 27, 2006

(54) Treatment of cell proliferative disorders with chlorotoxin

(76) Inventors: Vernon L. Alvarez, Birmingham, AL (US); Matthew A. Gonda, Birmingham, AL (US)

Correspondence Address: MORGAN LEWIS & BOCKIUS LLP, 1111 PENNSYLVANIA AVENUE NW, WASHINGTON, DC, 20004, US

(21) Appl. No.: 10/522,810

(22) PCT Filed: Jun. 2, 2003
(86) PCT No.: PCT/US03/17411

§ 371 Date: Sep. 20, 2005

(30) Foreign Application Priority Data
May 31, 2002 (US) 60384171
Aug. 27, 2002 (US) 60406033

(51) Int. Cl. A61K 38/08; C07K 7/06

(52) U.S. Cl. 514/16; 530/329; 530/328

(57) ABSTRACT

Disclosed is a method of treating cell proliferative disorders, such as cancer, with low doses of chlorotoxin and/or derivatives, analogs or fragments thereof, which are effective to inhibit or arrest abnormal cell growth.

### RELATED APPLICATIONS

This application claims the benefit of U.S. Provisional Application 60/406,033 (filed Aug. 27, 2002) and U.S. Provisional Application 60/384,171 (filed May 31, 2002) both of which are hereby incorporated by reference in their entirety.

### FIELD OF THE INVENTION

The present invention relates generally to the fields of cell physiology and oncology. More specifically, the present invention relates to a novel method of treating cell proliferative disorders, such as cancers, with doses of chlorotoxin and/or derivatives thereof which are effective to inhibit or arrest abnormal cell growth.

### BACKGROUND OF THE INVENTION

Tumors that originate in brain tissue are known as primary brain tumors as opposed to secondary brain tumors that develop when cancer metastasizes from other parts of the body to the brain. Primary brain tumors are classified by the type of tissue in which they begin. The most common brain tumors are gliomas, which begin in the glial (supportive) tissue. Astrocytomas are a type of glioma that arise from small, star-shaped cells called astrocytes. They may grow anywhere in the brain or spinal cord but most often arise in the cerebrum in adults and the brain stem, the cerebrum, and the cerebellum in children. A grade III astrocytoma is sometimes called anaplastic astrocytoma while a grade IV astrocytoma is usually called glioblastoma multiforme. Brain stem gliomas occur in the lowest, stemlike part of the brain. Tumors in this area generally cannot be removed. Most brain stem gliomas are high-grade astrocytomas. Ependymomas are a type of glioma that usually develop in the lining of the ventricles and may also occur in the spinal cord. Although these tumors can develop at any age, they are most common in childhood and adolescence. Oligodendrogliomas arise in the cells that produce myelin, the fatty covering that protects nerves. These rare tumors usually arise in the cerebrum, grow slowly, usually do not spread into surrounding brain tissue and occur most often in middle-aged adults but have been found in people of all ages.

There are other types of brain tumors that do not originate in glial tissue. Medulloblastomas were once thought to develop from glial cells. However, recent research suggests that these tumors develop from primitive (developing) nerve cells that normally do not remain in the body after birth. For this reason, medulloblastomas are sometimes called primitive neuroectodermal tumors. Most medulloblastomas arise in the cerebellum, however, they may occur in other areas as well. Meningiomas grow from the meninges and are usually benign. Because these tumors grow very slowly, the brain may be able to adjust to their presence and therefore these tumors often grow quite large before they cause symptoms. Schwannomas are benign tumors that begin in Schwann cells, which produce the myelin that protects the acoustic nerve. Acoustic neuromas are a type of schwannoma and occur mainly in adults. Craniopharyngiomas develop in the region of the pituitary gland near the hypothalamus and are usually benign, however, they are sometimes considered malignant because they can press on or damage the hypothalamus and affect vital functions. Germ cell tumors arise from primitive (developing) sex cells or germ cells. The most frequent type of germ cell tumor in the brain is the germinoma. Pineal region tumors occur in or around the pineal gland, a tiny organ near the center of the brain. The tumor can be slow growing (pineocytoma) or fast growing (pineoblastoma). The pineal region is very difficult to reach, and these tumors often cannot be removed.

Primitive neuroectodermal tumors are found both in the central and peripheral nervous systems. Primitive neuroectodermal tumors found only in the peripheral nervous system are referred to as peripheral primitive neuroectodermal tumors. Primitive neuroectodermal tumors manifest preferentially in children and have capacity for developing into a variety of neuronal, astrocytic, ependymal, muscular and melanotic lines. The conceptual basis of grouping these tumors together is based upon sharing common progenitor cells as well as sharing similar neoplastic transformations leading to tumors of similar morphological features and biological behavior. However, there remains controversy in placing all primitive neuroectodermal tumors into the same categories.

Supratentorial primitive neuroectodermal tumors include cerebral medulloblastomas, cerebral neuroblastomas, ependymoblastoma and other primitive neuroectodermal tumors, such as pineoblastomas. Peripheral neuroblastic tumors of the adrenal gland (medulla) and sympathetic nervous system are the most common type of childhood tumor outside of the central nervous system. Primary sites for these primitive neuroectodermal tumors are in the adrenals, abdominal, thoracic, cervical and pelvic sympathetic ganglia but include other primary sites as orbit, kidney, lung, skin, ovary, spermatic cord, and urinary bladder. Specific names of these related tumors are pheochromocytomas, paraganglioma, neuroblastomas, ganglioneuromas, ganglioneuroblastomas, neurofibromas, schwannomas, and malignant peripheral nerve sheath tumors. These all share common origin in the neural crest. Medulloblastomas are members of the primitive neuroectodermal tumors that are described as highly malignant embryonal tumors of the central nervous system found in the cerebellum.

Currently, surgery is the treatment of choice for tumors of the central nervous system. Surgery provides a definite diagnosis, relieves the mass bulkiness of the tumor and extends survival of the patient. The only post-surgery adjuvant treatment which is known to work effectively on central nervous system tumors is radiation, and it can prolong survival. Radiation treatment, however, has many undesirable side effects. It can damage the normal tissue of the patient, including the neuronal tissue. Radiation also can cause severe side effects (e.g., nausea, vomiting, hair loss).

The other common post-surgery adjuvant cancer treatment, chemotherapy, is relatively ineffective against neuroectodermal tumors. For example, chemotherapy against neuroectodermal tumors with nitrosourea agents is not curative. Many other cancer treating agents have been studied and tested, but generally they have a minimal effect on extending survival because many agents do not cross the blood-brain barrier. In view of these limited treatment options, the current prognosis for patients diagnosed with neuroectodermal tumors is not favorable. The median survival term for patients diagnosed with malignant astrocytomas having surgery and no adjuvant treatment is about fourteen weeks. Radiation therapy after surgery extends the median to about thirty-six weeks. The current two year survival rate for all forms of treatment is less than ten percent.

Other types of tumors are also difficult to combat by known cancer treatments. Lung cancer kills more Americans annually than the next four most frequently diagnosed neoplasms combined (Greenlee et al. (2001) CA Cancer J. Clin. 51, 15-36). Approximately eighty percent of primary lung tumors are of the non-small cell variety, which includes squamous cell and large cell carcinomas, as well as adenocarcinomas. Single-modality therapy is considered appropriate for most cases of early and late stage non-small cell lung cancer. Early stage tumors are potentially curable with surgery, chemotherapy, or radiotherapy, and late stage patients usually receive chemotherapy or best supportive care. Intermediate stage or locally advanced non-small cell lung cancer, which comprises twenty-five to thirty-five percent of all cases, is more typically treated with multi-modality therapy.

Breast cancer also presents treatment difficulties using known agents. The incidence of breast cancer in the United States has been rising at a rate of about two percent per year since 1980, and the American Cancer Society estimated that 192,000 cases of invasive breast cancer were diagnosed in 2001. Breast cancer is usually treated with surgery, radiotherapy, chemotherapy, hormone therapy or combinations of the various methods. A major reason for the failure of cancer chemotherapy in breast cancer is the development of resistance to the cytotoxic drugs. Combination therapy using drugs with different mechanisms of action is an accepted method of treatment which prevents development of resistance by the treated tumor. Anti-angiogenic agents are particularly useful in combination therapy because they are not likely to cause resistance development since they do not act on the tumor, but on normal host tissue.

Chlorotoxin is a thirty-six amino acid protein naturally derived from leiurus quinquestriatus scorpion venom (DeBin et al. (1993) Am. J. Physiol. 264: C361-369). Compositions (see U.S. Pat. Nos. 5,905,027 and 6,429,187 each of which is hereby incorporated by reference in their entirety) and methods (see U.S. Pat. Nos. 6,028,174 and 6,319,891 each of which is hereby incorporated by reference in their entirety) for diagnosing and treating neuroectodermal tumors (e.g., gliomas and meningiomas) have been developed based on the ability of chlorotoxin to bind to tumor cells of neuroectodermal origin (Soroceanu et al. (1998) Cancer Res. 58, 4871-4879; Ullrich et al. (1996) Neuroreport 7, 1020-1024; Ullrich et al. (1996) Am. J. Physiol. 270, C1511-C1521). Diagnosis of neuroectodermal tumors is accomplished by identification of labeled chlorotoxin bound to tumor cells while treatment of neuroectodermal tumors is accomplished by targeting tumors with cytotoxic agents linked to chlorotoxin. The present invention expands this area of therapeutics by providing a method for treating cell proliferative diseases, such as cancer, using chlorotoxin and/or derivatives thereof. The present invention also includes new toxin-derived therapeutic molecules.

### SUMMARY OF THE INVENTION

The present inventors have found that low doses of chlorotoxin and/or derivatives thereof, on the order of about 10-1,000 times lower than previously disclosed for the targeted treatment of glioma and meningioma and can inhibit or arrest the growth of tumor cells when said low doses of chlorotoxin and/or derivatives thereof, on the order of less than 2.0 mg/kg body weight of a subject are administered on their own. The present inventors have further found that these doses of chlorotoxin and/or derivatives thereof can effectively inhibit other types of cancer cells including, but not limited to, prostate cancer, breast cancer, and non-small cell lung carcinoma.

The invention encompasses an isolated polypeptide comprising a subunit of chlorotoxin or a related scorpion toxin, wherein the polypeptide specifically binds to a cancer cell. In some embodiments the isolated polypeptide comprises an amino acid sequence selected from the group consisting of SEQ ID NO: 6, 7, 8, 9, 10, 11, 12, 13 and 14. The invention includes toxin polypeptides comprising the amino acid sequence TTX₁X₂X₃MX₄X₅K (SEQ ID NO: 13), wherein X₁ is an acidic amino acid selected from the group consisting of aspartic acid and glutamic acid; X₂ is an amino acid selected from the group consisting of alanine, arginine, asparagine, aspartic acid, cysteine, glutamine, glutamic acid, glycine, histidine, isoleucine, leucine, lysine, proline, methionine, phenylalanine, serine, threonine, tryptophan, tyrosine and valine; X₃ is an amide amino acid selected from the group consisting of asparagine and glutamine; X₄ is an amino acid selected from the group consisting of serine, threonine and alanine; and X₅ is a basic amino acid selected from the group consisting of histine, lysine and arginine. In one embodiment, the isolated polypeptide comprises the amino acid sequence of SEQ ID NO: 14 (TTDHQMARK), SEQ ID NO: 94 (TTDQQMTKK) or SEQ ID NO: 95 (TTDPQMSKK).

The chlorotoxin and chlorotoxin derivatives of the invention bind to cancer cells selected from the group consisting of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma.

The invention also encompasses a fusion polypeptide comprising a subunit of chlorotoxin or a related scorpion toxin as described above, linked to a second polypeptide. In some embodiments, the second polypeptide comprises a cancer cell-binding domain which binds specifically of an epitope expressed only by a cancer cell. Examples of a cancer cell-binding domain include, but are not limited to, an antibody or fragment thereof. In other embodiments, the second polypeptide comprises a stabilization domain which prevent degradation of the fusion polypeptide. Examples of a stabilization domain include, but are not limited to, human serum albumin and transferrin. The stabilization domain can also include multimers of chlorotoxin or a chlorotoxin derivative, or chemical moieties such as PEG, poly-lysine, carbohydrates, fatty acids, and lipids.

In some embodiments of the invention, chlorotoxin or a chlorotoxin derivative is linked to a cytotoxic agent. Examples of cytoxic agents include, but are not limited to, gelonin, ricin, saponin, pseudonomas exotoxin, pokeweed antiviral protein, diphtheria toxin and complement proteins. In other embodiments, chlorotoxin or a chlorotoxin derivative is labeled. In a preferred embodiment the label is radioactive. The invention includes compositions comprising any of the chlorotoxin or chlorotoxin derivatives of the invention as described above.

The invention further includes a method of treating a disease characterized by abnormal cell proliferation in a mammal comprising administering the composition comprising the any of the chlorotoxin derivatives of the invention as described above. The invention also encompasses a method of treating a disease in a mammal characterized by abnormal cell proliferation comprising administering a composition consisting essentially of chlorotoxin or a related scorpion toxin. In a preferred embodiment, the composition is suitable for use in humans and the mammal is a human. In some embodiments, the disease is cancer. Types of cancer include, but are not limited to, lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma.

The methods of the invention include administration of chlorotoxin or a chlorotoxin derivative in an amount less than about 0.1 mg/kg body weight, preferably less than about 0.05 mg/kg body weight, even more preferably in a range of about 0.01 μg/kg body weight to about 0.1 mg/kg body weight, yet even more preferably about 0.1 μg/kg body weight to about 0.1 mg/kg body weight, including about 0.1 μg/kg body weight to about 0.05 mg/kg body weight, and most preferably in a range of about 0.1 μg/kg body weight to about 2.0 mg/kg body weight.

The invention further includes isolated nucleic acid molecule encoding the chlorotoxin polypeptide and chlorotoxin derivative polypeptides of the invention. In some embodiments, the nucleic acid molecule is operably linked to one or more expression control elements. The invention includes vectors comprising these nucleic acid molecules and host cells containing these vectors. In some embodiments, the host cell is a prokaryotic or eukaryotic host cells. The invention encompasses a method for producing a polypeptide comprising culturing these host cells under conditions in which the polypeptide encoded by said nucleic acid molecule is expressed.

### BRIEF DESCRIPTION OF THE DRAWINGS

FIG. 1 depicts a cytotoxicity assay in which low concentrations of chlorotoxin are shown to inhibit the growth and proliferation of glioblastoma cells.

FIG. 2 depicts the effect of four day incubation and wash-out on the ability of chlorotoxin to inhibit abnormal cell growth.

FIG. 3 depicts a cytotoxicity assay in which low concentrations of chlorotoxin are shown to inhibit the growth and proliferation of prostate cancer cells.

FIG. 4 depicts an in vivo assay of the ability of chlorotoxin to inhibit the growth of glioblastoma tumor cells in athymic nude mice.

FIG. 5 depicts an in vivo assay of the ability of chlorotoxin to enhance survival of athymic nude mice with intracranial glioblastoma tumors. Cessation of intravenous treatment indicated by arrow.

FIG. 6 depicts an in vivo assay of the ability of chlorotoxin to inhibit growth of glioblastoma tumors in the flanks of athymic nude mice.

FIG. 7 depicts a series of overlapping 10-mer peptides derived from chlorotoxin. Cysteine residues of SEQ ID NO: 1 are replaced in the 10-mers with serine to prevent cross-linking of peptides.

FIG. 8 depicts binding of chlorotoxin and 10-mer peptides 1-15.

FIG. 9 depicts binding of chlorotoxin and 10-mer peptides 16-27, 1, 5 and 10.

FIG. 10 depicts binding of peptide 21, the native core 9-mer, and each alanine-substituted 9-mer peptide to both U251 and PC3 cells.

FIG. 11 depicts binding of short scorpion toxins in PC3 human prostate cancer cells.

FIG. 12 depicts the effect of peptide 21 on the proliferation of D54MG cells was studied by adding increasing doses of peptide 21 to the cells and then measuring the uptake of ³H-thymidine.

### DETAILED DESCRIPTION OF THE INVENTION

It has been determined that low doses of chlorotoxin, on the order of about 10-1,000 times lower than previously disclosed for the treatment of glioma an meningioma, have a cytostatic effect on actively proliferating cells and can inhibit or arrest the growth of tumor cells without the addition of other exogenous therapeutic agents for the treatment of cell proliferative disorders, including, but not limited to, gliomas, meningiomas, prostate cancer, breast cancer and non-small cell lung carcinomas. Furthermore, polypeptide derivatives of chlorotoxin containing one or more of the regions disclosed herein can be used for the treatment of cell proliferative disorders in a manner similar to native chlorotoxin.

Inhibition or arrest of cell proliferation associated with a disease, such as cancer, can serve to enhance natural defenses within the subject. For example, arresting or inhibiting the growth of cancer cells enhances the ability of the immune system to mount a more effective response to the cancer. Further, arresting or inhibiting cell proliferation can slow angiogenesis, starving the tumor of nutrients and further enhances the killing or elimination of the tumor.

Chlorotoxin and Derivative Polypeptides

This invention includes a pharmaceutical composition for the treatment of abnormal cell growth or for a disease characterized by cell proliferation in a mammal, including a human, comprising, consisting essentially of, or consisting of an amount of chlorotoxin and/or derivatives thereof that is effective in inhibiting abnormal cell growth and a pharmaceutically acceptable carrier. As used herein, the terms “abnormal cell growth” and “disease characterized by cell proliferation” unless otherwise indicated, refer to cell growth that is independent of normal regulatory mechanisms (e.g., loss of contact inhibition). This includes the abnormal growth and/or proliferation of cells in both benign and malignant cells of neoplastic diseases. Inhibition of abnormal cell growth can occur by a variety of mechanisms including, but not limited to, cell death, apoptosis, arrest of mitosis, inhibition of cell division, transcription, translation, transduction, etc.

As used herein, an “effective amount” of chlorotoxin is an amount which exerts an effect on cells exhibiting abnormal growth, such as cancer cells. As used herein, compositions comprising, treatment with or administration of “chlorotoxin” includes to the same degree treatment with chlorotoxin analogues, derivatives, fragments, variants, related peptides and mimetics disclosed herein.

In one embodiment of the composition and methods of the invention, the abnormal cell growth is cancer. As used herein, the term “cancer” unless otherwise indicated, refers to diseases that are characterized by uncontrolled, abnormal cell growth and/or proliferation. Types of cancer where the compositions are useful include, but are not limited to, prostate cancer, breast cancer, lung cancer, non-small cell lung carcinoma, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancers, spinal axis tumors, glioma, meningioma, pituitary adenoma, or a combination of one or more of the foregoing cancers.

In another embodiment of the compositions and methods of the invention, the abnormal cell growth is a benign proliferative disease, including, but not limited to, benign prostatic hyperplasia, hypertrophy or restinosis.

As discussed above, the invention includes compositions and methods for the treatment of abnormal cell growth in a mammal, including a human, which comprises, consists of, or consists essentially of an effective amount of a chlorotoxin, in combination with a pharmaceutically acceptable carrier.

As used herein, the term “chlorotoxin” unless otherwise described, refers to the full-length, thirty-six amino acid polypeptide naturally derived from Leiurus quinquestriatus scorpion venom (DeBin et al. (1993) Am. S. Physiol. 264, C361-369) which comprises the amino acid sequence of native chlorotoxin as set forth in SEQ ID NO: 1. The term “chlorotoxin” includes polypeptides comprising SEQ ID NO: 1 which have been synthetically or recombinantly produced, such as those disclosed in U.S. Pat. No. 6,319,891, which is herein incorporated by reference in its entirety.

As used herein, the term “chlorotoxin subunit” or “subunit of chlorotoxin” refers to a peptide comprising less than thirty-six contiguous amino acids of chlorotoxin and which is capable of specifically binding to cancer cells.

As used herein, the term “chlorotoxin derivative” refers to derivatives, analogs, variants, polypeptide fragments and mimetics of chlorotoxin and related peptides which retain the same activity as chlorotoxin, such as binding specifically binding to a cancer cell when compared to a normal cell, can also be used for practicing the methods of the invention. Examples of derivatives include, but are not limited to, peptide variants of chlorotoxin, peptide fragments of chlorotoxin, for example, fragments comprising or consisting of contiguous 10-mer peptides of SEQ ID NO: 1, 2, 3, 4, 5, 6 or 7 or comprising about residues 10-18 or 21-30 of SEQ ID NO: 1, core binding sequences, and peptide mimetics.

Chlorotoxin and peptide derivatives thereof can be prepared using standard solid phase (or solution phase) peptide synthesis methods, as is known in the art. In addition, the nucleic acids encoding these peptides may be synthesized using commercially available oligonucleotide synthesis instrumentation and produced recombinantly using standard recombinant production systems. The production using solid phase peptide synthesis is necessitated if non-gene-encoded amino acids are to be included. The term “chlorotoxin derivative” as used herein is synonymous with “variant” also includes modifications to the chlorotoxin sequence by one or more deletions of up to 10 (e.g., 1 to 7 or 1 to 5 amino acids; insertions of a total of up to 10 (e.g., 1 to 5) amino acids internally within the amino acid sequence of chlorotoxin; or of up to a total of 100 amino acids at either terminus of the chlorotoxin sequence; or conservative substitutions of a total of up to 15 (e.g., 1 to 5) amino acids.

Derivatives of chlorotoxin include polypeptides comprising a conservative or non-conservative substitution of at least one amino acid residue when the derivative sequence and the chlorotoxin sequence are maximally aligned. The substitution may be one which enhances at least one property or function of chlorotoxin, inhibits at least one property or function of chlorotoxin, or is neutral to at least one property or function of chlorotoxin. As used herein, a “property or function” of chlorotoxin includes, but is not limited to, at least one selected from the group consisting of the ability to arrest abnormal cell growth, cause paralysis of a subject, specific binding to a benign or malignant cancer cell when compared to a non-cancer cell (i.e., normal), and killing of a benign or malignant cancer cell. In terms of the present disclosure, the cancer cell may be in vivo, ex vivo, in vitro, a primary isolate from a subject, a cultured cell or a cell line.

Derivatives of chlorotoxin further include polypeptides comprising the amino acid sequence KGRGKSY (SEQ ID NO: 8), corresponding to amino acid residues 23-29 of SEQ ID NO: 1. Derivatives of chlorotoxin also include polypeptides comprising the amino acid sequence TTX₁X₂X₃MX₄X₅K (SEQ ID NO: 13) corresponding to amino acid residues 7-15 of SEQ ID NO: 1, wherein X₁ is an acidic amino acid selected from the group consisting of aspartic acid and glutamic acid; X₂ is an amino acid selected from the group consisting of alanine, arginine, asparagine, aspartic acid, cysteine, glutamine, glutamic acid, glycine, histidine, isoleucine, leucine, lysine, proline, methionine, phenylalanine, serine, threonine, tryptophan, tyrosine and valine; X₃ is an amide amino acid selected from the group consisting of asparagine and glutamine; X₄ is an any amino acid but in a preferred embodiment is selected from the group consisting of serine, threonine and alanine; and X₅ is a basic amino acid selected from the group consisting of histine, lysine and arginine. In one embodiment, X₁ is aspartic acid, X₂ is histidine or proline, X₃ is glutamine, X₄ is alanine and X₅ is arginine or lysine.

Peptide variants of chlorotoxin include, but are not limited to, deletion or conservative amino acid substitution variants of SEQ ID NO: 1. As used herein, a conservative variant refers to alterations in the amino acid sequence that do not adversely substantially affect the biological functions of the peptide. A substitution, insertion or deletion is said to adversely affect the peptide when the altered sequence substantially prevents or disrupts a biological function associated with the peptide (e.g., binding to a cancer cell). For example, the overall charge, structure or hydrophobic/hydrophilic properties of the peptide can be altered without adversely affecting a biological activity. Accordingly, the amino acid sequence can be altered, for example to render the peptide more hydrophobic or hydrophilic, without adversely affecting the biological activities of the peptide.

The methods of the invention include corresponding polypeptide toxins of other scorpion species that display similar or related activity to chlorotoxin for the diagnosis and treatment of diseases associated with abnormal cell proliferation as described herein, including cancer. For purposes of the specification, “similar or related activity to chlorotoxin” is defined as binding to cells displaying abnormal cell growth, including benign cells exhibiting abnormal growth and malignant cancer cells. Examples of such polypeptide toxins include, but are not limited to, toxins which contain one or more of the binding domains of chlorotoxin set forth in SEQ ID NO: 8 or SEQ ID NO: 13, and any of the consensus sequences set forth in Table 1.

TABLE 1
Scorpion toxin alignments (sequence identifier in parenthesis)

Small Toxin (Peptide I) - Mesobuthus tamulus sindicus

| | | |
|---|---|---|
| | | 1 36 |
| Chlorotoxin | (SEQ ID NO: 1) | MCMPCFTTDHQMARKCDDCCGGKGRGKCYGPQCLGR |
| Small Toxin | (SEQ ID NO: 15) | RCKPCFTTDPQMSKKCADCCGGKGKGKCYGPQCLC- |
| Consensus | (SEQ ID NO: 16) | C PCFTTD QMAKKC DCCGGKGKGKCYGPQCLC |

Probable Toxin LQH 8/6 - Leiurus quinquestriatus hebraeus

| | | |
|---|---|---|
| | | 1 38 |
| Chlorotoxin | (SEQ ID NO: 1) | MCMPCFTTDHQMARKCDDCCGGKGRGKCYGPQCLCR-- |
| Toxin LQH | (SEQ ID NO: 17) | RCSPCFTTDQQMTKKCYDCCGGKGKGKCYGPQCICAPY |
| Consensus | (SEQ ID NO: 18) | C PCFTTD QM KKC DCCGGKGKGKCYGPQCIC |

Chinese Scorpion - Mesobuthus martensii

| | | |
|---|---|---|
| | | 1 35 |
| Chlorotoxin | (SEQ ID NO: 1) | -----------------------MCMPCFTTDHQM |
| Chinese | (SEQ ID NO: 19) | MKFLYGIVFIALFLTVMFATQTDGCGPCFTTDANM |
| Consensus | (SEQ ID NO: 20) | C PCFTTD NM |
| | | 36 61 |
| Chlorotoxin | (SEQ ID NO: 1) | ARKCDDCCGGKGRGKCYGPQCLCR-- |
| Chinese | (SEQ ID NO: 19) | ARKCRECCGGIG--KCFGPQCLCNRI |
| Consensus | (SEQ ID NO: 20) | ARKC DCCGG G KCFGPQCLC |
| | | 1 35 |
| Chlorotoxin | (SEQ ID NO: 1) | -----------------------MCMPCFTTDHQM |
| Chinese | (SEQ ID NO: 21) | MKFLYGIVFIALFLTVMFATQTDGCGPCFTTDANM |
| Consensus | (SEQ ID NO: 22) | C PCFTTD NM |
| | | 36 59 |
| Chlorotoxin | (SEQ ID NO: 1) | ARKCDDCCGGKGRGKCYGPQCLCR |
| Chinese | (SEQ ID NO: 21) | ARKCRECCGGIGKCFGPQCLCNRI |
| Consensus | (SEQ ID NO: 22) | ARKC DCCGG GK C |

Insect toxin I5 (lesser Asian Scorpion) - Mesobuthus eupeus

| | | |
|---|---|---|
| | | 1 37 |
| Chlorotoxin | (SEQ ID NO: 1) | MCMPCFTTDHQMARKCDDCCGGKGRGKCYGPQCLCR- |
| toxin I5 | (SEQ ID NO: 23) | MCMPCFTTDPNMANKCRDCCGG-GK-KCFGPQCLCNR |
| Consensus | (SEQ ID NO: 24) | MCMPCFTTD NMA KC DCCGG GK KCFGPQCLC |
| | | 1 36 |
| Chlorotoxin | (SEQ ID NO: 1) | MCMPCFTTDHQMARKCDDCCGGKGRGKCYGPQCLCR |
| toxin I5 | (SEQ ID NO: 25) | MCMPCFTTDPNMANKCRDCCGGGKKCFGPQCLCNR- |
| Consensus | (SEQ ID NO: 26) | MCMPCFTTD NMA KC DCCGG K C |

Insectotoxin I1 (lesser Asian Scorpion) - Mesobuthus eupeus

| | | |
|---|---|---|
| | | 1 38 |
| Chlorotoxin | (SEQ ID NO: 1) | MCMPCFTTDHQMARKCDDCCGGKGRGKCYGPQCLCR-- |
| toxin I1 | (SEQ ID NO: 27) | MCMPCFTTRPDMAQQCRACCKG--RGKCFGPQCLCGYD |
| Consensus | (SEQ ID NO: 28) | MCMPCFTT MA C CC G RGKCFGPQCLC |
| | | 1 36 |
| Chlorotoxin | (SEQ ID NO: 1) | MCMPCFTTDHQMARKCDDCCGGKGRGKCYGPQCLCR |
| toxin I1 | (SEQ ID NO: 29) | MCMPCFTTRPDMAQQCRACCKGRGKCFGPQCLCGYD |
| Consensus | (SEQ ID NO: 30) | MCMPCFTT MA C CC GKGK C |

Insectotoxin 15A (lesser Asian Scorpion) - Mesobuthus eupeus

| | | |
|---|---|---|
| | | 1 37 |
| Chlorotoxin | (SEQ ID NO: 1) | MCMPCFTTDHQMARKCDDCCGGKGRGKCYGPQCLCR- |
| toxin 15A | (SEQ ID NO: 31) | MCMPCFTTDPNMAKKCRDCCGGNG--KCFGPQCLCNR |
| Consensus | (SEQ ID NO: 32) | MCMPCFTTD NMAKKC DCCGG G KCFGPQCLC |
| | | 1 36 |
| Chlorotoxin | (SEQ ID NO: 1) | MCMPCFTTDHQMARKCDDCCGGKGRGKCYGPQCLCR |
| toxin 15A | (SEQ ID NO: 33) | MCMPCFTTDPNMAKKCRDCCGGNGKCFGPQCLCNR- |
| Consensus | (SEQ ID NO: 34) | MCMPCFTTD NMAKKC DCCGG GK C |

Neurotoxin P2 (Moroccan scorpion) - Androctonus mauretanicus

| | | |
|---|---|---|
| | | 1 38 |
| Chlorotoxin | (SEQ ID NO: 1) | MCMPCFTTDHQMARKCDDCCGGKGRGKCYGPQCLCR-- |
| Neurotoxin | (SEQ ID NO: 35) | -CGPCFTTDPYTESKCATCCGG--RGKCVGPQCLCNRI |
| Consensus | (SEQ ID NO: 36) | C PCFTTD KC CCGG RGKC GPQCLC |
| | | 1 36 |
| Chlorotoxin | (SEQ ID NO: 1) | MCMPCFTTDHQMARKCDDCCGGKGRGKCYGPQCLCR |
| Neurotoxin | (SEQ ID NO: 37) | -CGPCFTTDPYTESKCATCCGGRGKCVGPQCLCNRI |
| Consensus | (SEQ ID NO: 38) | C PCFTTD KC CCGGKGK C |

Alignment of Chlorotoxin with all above scorpion toxins

| | |
|---|---|
| | 1 50 |
| Chlorotoxin | -----------------------MCMPCFTTDHQMARKCDDCCGGKGRGK |
| Small Toxin | -----------------------RCKPCFTTDPQMSKKCADCCGGKGKGK |
| Toxin LQH | -----------------------RCSPCFTTDQQMTKKCYDCCGGKGKGK |
| Chinese | MKFLYGIVFIALFLTVMFATQTDGCGPCFTTDANMARKCRECCGGIG--K |
| toxin I5 | -----------------------MCMPCFTTDPNMANKCRDCCGGGK--K |
| toxin I1 | -----------------------MCMPCFTTRPDMAQQCRACCKGRG--K |
| toxin 15A | -----------------------MCMPCFTTDPNMAKKCRDCCGGNG--K |
| Neurotoxin | ------------------------CGPCFTTDPYTESKCATCCGGRG--K |
| Consensus | MCMPCFTTDPNMAKKCRDCCGGKG K |

| | | |
|---|---|---|
| | | 51 61 |
| Chlorotoxin | (SEQ ID NO: 1) | CYGPQCLCR-- |
| Small Toxin | (SEQ ID NO: 15) | CYGPQCLC--- |
| Toxin LQH | (SEQ ID NO: 17) | CYGPQCICAPY |
| Chinese | (SEQ ID NO: 19) | CFGPQCLCNRI |
| toxin I5 | (SEQ ID NO: 25) | CFGPQCLCNR- |
| toxin I1 | (SEQ ID NO: 29) | CFGPQCLCGYD |
| toxin 15A | (SEQ ID NO: 33) | CFGPQCLCNR- |
| Neurotoxin | (SEQ ID NO: 37) | CVGPQCLCNRI |
| Consensus | (SEQ ID NO: 39) | CFGPQCLCNR |

| | |
|---|---|
| Alignment of Chlorotoxin with toxins not requiring gaps to align | +TL,4L5 |

| | | |
|---|---|---|
| | | 1 38 |
| Chlorotoxin | (SEQ ID NO: 1) | MCMPCFTTDHQMARKCDDCCGGKGRGKCYGPQCLCR-- |
| Small Toxin | (SEQ ID NO: 15) | RCKPCFTTDPQMSKKCADCCGGKGKGKCYGPQCLC--- |
| Toxin LQH | (SEQ ID NO: 17) | RCSPCFTTDQQMTKKCYDCCGGKGKGKCYGPQCICAPY |
| Consensus | (SEQ ID NO: 40) | RC PCFTTD QMSKKC DCCGGKGKGKCYGPQCLC |

Alignment of Chlorotoxin with toxins requiring gaps (gaps removed)

| | | |
|---|---|---|
| | | 1 50 |
| Chlorotoxin | -----------------------MCMPCFTTDHQMARKCDDCCGGKGRGK | |
| CT-Neurotox | MKFLYGIVFIALFLTVMFATQTDGCGPCFTTDANMARKCRECCGGIGKCF | |
| toxin I5 | -----------------------MCMPCFTTDPNMANKCRDCCGGGKKCF | |
| toxin I1 | -----------------------MCMPCFTTRPDMAQQCRACCKGRGKCF | |
| toxin 15A | -----------------------MCMPCFTTDPNMAKKCRDCCGGNGKCF | |
| Neurotoxin | ------------------------CGPCFTTDPYTESKCATCCGGRGKCV | |
| Consensus | MCMPCFTTDPNMARKCRDCCGGRGKCF | |
| | | 51 |
| Chlorotoxin | (SEQ ID NO: 1) | CYGPQCLCR |
| CT-Neurotox | (SEQ ID NO: 19) | GPQCLCNRI |
| toxin I5 | (SEQ ID NO: 25) | GPQCLCNR- |
| toxin I1 | (SEQ ID NO: 29) | GPQCLCGYD |
| toxin 15A | (SEQ ID NO: 33) | GPQCLCNR- |
| Neurotoxin | (SEQ ID NO: 37) | GPQCLCNRI |
| Consensus | (SEQ ID NO: 41) | GPQCLCNR |

Chlorotoxin Peptide 8 alignment with other scorpion toxins

| | | |
|---|---|---|
| | | 1 |
| Pep8-Ctlx | (SEQ ID NO: 42) | CGGKGRGKCY |
| Pep8-SCX1_BUTSI | (SEQ ID NO: 43) | CGGKGKGKCY |
| Consensus | (SEQ ID NO: 43) | CGGKGKGKCY |
| | | 1 |
| Pep8-Ctlx | (SEQ ID NO: 42) | CGGKGRGKCY |
| Pep8-SCX8_LEIQH | (SEQ ID NO: 43) | CGGKGKGKCY |
| Consensus | (SEQ ID NO: 43) | CGGKGKGKCY |
| | | 1 12 |
| Pep8-Ctlx | (SEQ ID NO: 42) | CGGKGRGKCY-- |
| Pep8-AF079059_2 | (SEQ ID NO: 44) | CGGIG--KCFGP |
| Consensus | (SEQ ID NO: 45) | CGG GRGKCFGP |
| | | 1 |
| Pep8-Ctlx | (SEQ ID NO: 42) | CGGKGRGKCY |
| Pep8-AF079059_2 | (SEQ ID NO: 44) | CGGIGKCFGP |
| Consensus | (SEQ ID NO: 46) | CGG GK |
| | | 1 12 |
| Pep8-Ctlx | (SEQ ID NO: 42) | CGGKGRGKCY-- |
| Pep8-JN0361 | (SEQ ID NO: 47) | CGG-GK-KCFGP |
| Consensus | (SEQ ID NO: 48) | CGGKGKGKCFGP |
| | | 1 |
| Pep8-Ctlx | (SEQ ID NO: 42) | CGGKGRGKCY |
| Pep8-JN0361 | (SEQ ID NO: 47) | CGGGKKCFGP |
| Consensus | (SEQ ID NO: 49) | CGG K |
| | | 1 12 |
| Pep8-Ctlx | (SEQ ID NO: 42) | CGGKGRGKCY-- |
| Pep8-SCX1_BUTEU | (SEQ ID NO: 50) | --CKGRGKCFGP |
| Consensus | (SEQ ID NO: 51) | CG KGRGKCFGP |
| | | 1 |
| Pep8-Ctlx | (SEQ ID NO: 42) | CGGKGRGKCY |
| Pep8-SCX1_BUTEU | (SEQ ID NO: 50) | CKGRGKCFGP |
| Consensus | (SEQ ID NO: 52) | C GKGK |
| | | 1 12 |
| Pep8-Ctlx | (SEQ ID NO: 42) | CGGKGRGKCY-- |
| Pep8-SCX5_BUTEU | (SEQ ID NO: 53) | CGGNG--KCFGP |
| Consensus | (SEQ ID NO: 54) | CGG GRGKCFGP |
| | | 1 |
| Pep8-Ctlx | (SEQ ID NO: 42) | CGGKGRGKCY |
| Pep8-SCX5_BUTEU | (SEQ ID NO: 53) | CGGNGKCFGP |
| Consensus | (SEQ ID NO: 55) | CGG GK |
| | | 1 12 |
| Pep8-Ctlx | (SEQ ID NO: 42) | CGGKGRGKCY-- |
| Pep8-SCXP_ANDMA | (SEQ ID NO: 56) | CGG--RGKCVGP |
| Consensus | (SEQ ID NO: 57) | CGGKGRGKC GP |
| | | 1 |
| Pep8-Ctlx | (SEQ ID NO: 42) | CGGKGRGKCY |
| Pep8-SCXP_ANDMA | (SEQ ID NO: 56) | CGGRGKCVGP |
| Consensus | (SEQ ID NO: 58) | CGGKGK |
| | | 1 12 |
| Pep8-Ctlx | (SEQ ID NO: 42) | CGGKGRGKCY-- |
| Pep8-SCX1_BUTSI | (SEQ ID NO: 43) | CGGKGKGKCY-- |
| Pep8-SCX8_LEIQH | (SEQ ID NO: 43) | CGGKGKGKCY-- |
| Pep8-AF079059_2 | (SEQ ID NO: 44) | CGG--IGKCFGP |
| Pep8-JN0361 | (SEQ ID NO: 47) | CGG--GKKCFGP |
| Pep8-SCX1_BUTEU | (SEQ ID NO: 50) | CKG--RGKCFGP |
| Pep8-SCX5_BUTEU | (SEQ ID NO: 53) | CGG--NGKCFGP |
| Pep8-SCXP_ANDMA | (SEQ ID NO: 56) | CGG--RGKCVGP |
| Consensus | (SEQ ID NO: 59) | CGG RGKCFGP |
| | | 1 |
| Pep8-Ctlx | (SEQ ID NO: 42) | CGGKGRGKCY |
| Pep8-SCX1_BUTSI | (SEQ ID NO: 43) | CGGKGKGKCY |
| Pep8-SCX8_LEIQH | (SEQ ID NO: 43) | CGGKGKGKCY |
| Pep8-AF079059_2 | (SEQ ID NO: 44) | CGGIGKCFGP |
| Pep8-JN0361 | (SEQ ID NO: 47) | CGGGKKCFGP |
| Pep8-SCX1_BUTEU | (SEQ ID NO: 50) | CKGRGKCFGP |
| Pep8-SCX5_BUTEU | (SEQ ID NO: 53) | CGGNGKCFGP |
| Pep8-SCXP_ANDMA | (SEQ ID NO: 56) | CGGRGKCVGP |
| Consensus | (SEQ ID NO: 60) | CGGKGKCFGP |

Chlorotoxin Peptide 21 alignment with other scorpion toxins

| | | |
|---|---|---|
| | | 1 |
| Pep21-cltx | (SEQ ID NO: 61) | TTDHQMARKC |
| Pep21-SCX1_BUTSI | (SEQ ID NO: 62) | TTDPQMSKKC |
| Consensus | (SEQ ID NO: 63) | TTD QMAKKC |
| | | 1 |
| Pep21-cltx | (SEQ ID NO: 61) | TTDHQMARKC |
| Pep21-SCX8_LEIQH | (SEQ ID NO: 64) | TTDQQMTKKC |
| Consensus | (SEQ ID NO: 65) | TTD QM KKC |
| | | 1 |
| Pep21-cltx | (SEQ ID NO: 61) | TTDHQMARKC |
| Pep21-AF079059_2 | (SEQ ID NO: 66) | TTDANMARKC |
| Consensus | (SEQ ID NO: 67) | TTD NMARKC |
| | | 1 |
| Pep21-cltx | (SEQ ID NO: 61) | TTDHQMARKC |
| Pep21-JN0361 | (SEQ ID NO: 68) | TTDPNMANKC |
| Consensus | (SEQ ID NO: 69) | TTD NMA KC |
| | | 1 |
| Pep21-cltx | (SEQ ID NO: 61) | TTDHQMARKC |
| Pep21-SCX1_BUTEU | (SEQ ID NO: 70) | TTRPDMAQQC |
| Consensus | (SEQ ID NO: 71) | TT MA C |
| | | 1 |
| Pep21-cltx | (SEQ ID NO: 61) | TTDHQMARKC |
| Pep21-SCX5_BUTEU | (SEQ ID NO: 72) | TTDPNMAKKC |
| Consensus | (SEQ ID NO: 73) | TTD NMAKKC |
| | | 1 |
| Pep21-cltx | (SEQ ID NO: 61) | TTDHQMARKC |
| Pep21-SCXP_ANDMA | (SEQ ID NO: 74) | TTDPYTESKC |
| Consensus | (SEQ ID NO: 75) | TTD KC |
| | | 1 |
| Pep21-cltx | (SEQ ID NO: 61) | TTDHQMARKC |
| Pep21-SCX1_BUTSI | (SEQ ID NO: 62) | TTDPQMSKKC |
| Pep21-SCX8_LEIQH | (SEQ ID NO: 64) | TTDQQMTKKC |
| Pep21-AF079059_2 | (SEQ ID NO: 66) | TTDANMARKC |
| Pep21-JN0361 | (SEQ ID NO: 68) | TTDPNMANKC |
| Pep21-SCX1_BUTEU | (SEQ ID NO: 70) | TTRPDMAQQC |
| Pep21-SCX5_BUTEU | (SEQ ID NO: 72) | TTDPNMAKKC |
| Pep21-SCXP_ANDMA | (SEQ ID NO: 74) | TTDPYTESKC |
| Consensus | (SEQ ID NO: 76) | TTDPNMAKKC |

As used herein, the term “related scorpion toxin” refers to any of the toxins or related peptides, such as those disclosed in Table 1, displaying amino acid and/or nucleotide sequence identity to chlorotoxin. Examples of related scorpion toxins include, but are not limited to, CT neurotoxin from Mesobuthus martensii (GenBank Accession AAD47373), Neurotoxin BmK 41-2 from Buthus martensii karsch (GenBank Accession A59356), Neurotoxin Bm12-b from Buthus martensii (GenBank Accession AAK16444), Probable Toxin LQH 8/6 from Leiurus quinquestriatus hebraeu (GenBank Accession P55966), Small toxin from Mesobuthus tamulus sindicus (GenBank Accession P15229), the sequences of which are all herein incorporated by reference in their entirety.

Homology or sequence identity at the nucleotide or amino acid sequence level is determined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference), recommended for query sequences over eighty-five nucleotides or amino acids in length.

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink-1 (generates word hits at every wink^{th} position along the query); and gapw-16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The present invention encompasses the allelic variants, conservative substitution variants, and the members of the scorpion toxin peptide family, having an amino acid sequence of at least about seventy-five percent, at least about eighty-five percent, at least about ninety percent sequence, at least about ninety-five percent, or at least about ninety-nine percent sequence identity with the entire chlorotoxin sequence set forth in SEQ ID NO: 1. Identity or homology with respect to such sequences is defined herein as the percentage of amino acid residues in the candidate sequence that are identical with the known peptides, after alignment the sequences.

Fusion proteins, or N-terminal, C-terminal or internal extensions, deletions, or insertions into the peptide sequence shall not be construed as affecting homology. Examples of such extensions include, but are not limited to, the following sequences:

(SEQ ID NO: 2)

HHHHHHMCMPCFTTDHQMARKCDDCCGGKGRGKCYGPQCLCR,

(SEQ ID NO: 3)

YMCMPCFTTDHQMARKCDDCCGGKGRGKCYGPQCLCR,

(SEQ ID NO: 4)

YSYMCMPCFTTDHQMARKCDDCCGGKGRGKCYGPQCLCR.

The chlorotoxin peptide variants include peptides having a fragment of the amino acid sequence set forth in SEQ ID NO: 1, having at least about 7, 8, 9, 10, 15, 20, 25, 30, or 35 contiguous amino acid residues. The peptide variants further include those fragments associated with the activity of chlorotoxin. Such fragments, also referred to as polypeptides, may contain functional regions of the chlorotoxin peptide identified as regions of the amino acid sequence which correspond to known peptide domains, as well as regions of pronounced hydrophilicity. Variants may also include peptide with at least two core sequences linked to one another, in any order, with intervening amino acids removed or replaced by a linker sequence. The regions are all easily identifiable by using commonly available protein sequence analysis software such as Mac Vector (Oxford Molecular).

Contemplated peptide variants further include those containing predetermined mutations by, e.g., homologous recombination, site-directed or PCR mutagenesis, and the alleles or other naturally occurring variants of the family of peptides; and derivatives wherein the peptide has been covalently modified by substitution, chemical, enzymatic or other appropriate means with a moiety other than a naturally occurring amino acid (for example a detectable moiety such as an enzyme or radioisotope). Examples of chlorotoxin variant peptides include, but are not limited to the following sequences:

(SEQ ID NO: 5)

MCMPCFTTDHQMARKCDDCCGGKGRGKCFGPQCLCR,

(SEQ ID NO: 6)

RCKPCFTTDPQMSKKCADCCGGKGKGKCYGPQCLC,

(SEQ ID NO: 7)

RCSPCFTTDQQMTKKCYDCCGGKGKGKCYGPQCICAPY.

Nucleic Acids Encoding Chlorotoxin and Chlorotoxin Derivatives

The present invention further provides nucleic acid molecules that encode chlorotoxin and chlorotoxin derivative polypeptides of the invention. Such nucleic acid molecules can be in an isolated form, or can be operably linked to expression control elements or vector sequences. The present invention further provides host cells that contain the vectors via transformation, transfection, electroporation or any other art recognized means of introducing a nucleic acid into a cell.

As used herein, a “replicon” is any genetic element (e.g., plasmid, chromosome, virus) that functions as an autonomous unit of DNA replication in vivo (i.e., capable of replication under its own control).

As used herein, a “vector” is a replicon, such as plasmid, phage or cosmid, to which another nucleic acid (e.g., DNA) segment may be attached so as to bring about the replication of the attached segment. Vectors of the invention include viral vectors.

As used herein, a “nucleic acid” refers to the polymeric form of ribonucleotide or deoxyribonucleotides (adenine, guanine, thymine, and/or cytosine) in either its single stranded form, or in double-stranded helix. This term refers only to the primary and secondary structure of the molecule and is not limited to any particular tertiary form. Thus, this term includes single-stranded RNA or DNA, double-stranded DNA found in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (e.g., the strand having a sequence homologous to the mRNA).

A nucleic acid “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

As used herein, a “promoter sequence” is a DNA regulatory region capable of binding RNA polymerase in a cell and initiating transcription of a downstream (3′ direction) coding sequence. For purposes of defining the present invention, the promoter sequence is bounded (inclusively) at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes.

A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence or a signal peptide sequence from a scorpion toxin may be used. This sequence encodes a signal peptide, N-terminal to the polypeptide, that communicates to the host cell to direct the polypeptide to the cell surface or secrete the polypeptide into the media. This signal peptide is clipped off by the host cell before the protein leaves the cell. Signal sequences can be found associated with a variety of proteins native to prokaryotes and eukaryotes. For instance, alpha-factor, a native yeast protein, is secreted from yeast, and its signal sequence can be attached to heterologous proteins to be secreted into the media (see U.S. Pat. No. 4,546,082). Further, the alpha-factor and its analogs have been found to secrete heterologous proteins from a variety of yeast, such as Saccharomyces and Kluyveromyces (EP 88312306.9; EP 0324274 publication, and EP 0301669). An example for use in mammalian cells is the tPA signal used for expressing Factor VIIIc light chain.

A cell has been “transformed” by a exogenous or heterologous nucleic acid when such nucleic acid as been introduced inside the cell. The transforming nucleic acid may or may not be integrated (covalently linked) into chromosomal DNA making up the genome of the cell. In prokaryotes, for example, the transforming nucleic acid may be maintained on an episomal element such as a plasmid or viral vector. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming nucleic acid.

As used herein, a “cell line” is a clone of a primary cell that is capable of stable growth in vitro for many generations. As used herein, nucleic acid sequences display “substantial identity” when at least about 85% (preferably at least about 90% and most preferably at least about 95%/o) of the nucleotides match over the defined length of the nucleotide sequences. Sequences that are substantially identical can be identified in a Southern hybridization experiment under, for example, stringent conditions as defined for that particular system. Defining appropriate hybridization conditions is within the skill of the art.

A “heterologous” region of the nucleic acid construct is an identifiable segment of a nucleic acid within a larger nucleic acid molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g. a cDNA where the genomic coding sequence contains introns, or synthetic sequences having codons different than the native gene).

Vectors are used to simplify manipulation of the nucleic acids which encode the chlorotoxin and chlorotoxin derivative polypeptides, either for preparation of large quantities of nucleic acids for further processing (cloning vectors) or for expression of the polypeptides (expression vectors). Vectors comprise plasmids, viruses (including phage), and integrated DNA fragments (i.e., fragments that are integrated into the host genome by recombination). Cloning vectors need not contain expression control sequences. However, control sequences in an expression vector include transcriptional and translational control sequences such as a transcriptional promoter, a sequence encoding suitable ribosome binding sites, and sequences which control termination of transcription and translation. The expression vector should preferably include a selection gene to facilitate the stable expression of the chlorotoxin gene and/or to identify transformed cells. However, the selection gene for maintaining expression can be supplied by a separate vector in co-transformation systems using eukaryotic host cells.

Suitable vectors generally will contain replicon (origins of replication, for use in non-integrative vectors) and control sequences which are derived from species compatible with the intended expression host. By the term “replicable” vector as used herein, it is intended to encompass vectors containing such replicons as well as vectors which are replicated by integration into the host genome. Transformed host cells are cells which have been transformed or transfected with vectors containing chlorotoxin or chlorotoxin derivative polypeptide encoding nucleic acid. The expressed polypeptides may be secreted into the culture supernatant, under the control of suitable processing signals in the expressed peptide (e.g. homologous or heterologous signal sequences).

Expression vectors for host cells ordinarily include an origin of replication, a promoter located upstream from the chlorotoxin or chlorotoxin derivative polypeptide coding sequence, together with a ribosome binding site, a polyadenylation site, and a transcriptional termination sequence. Those of ordinary skill will appreciate that certain of these sequences are not required for expression in certain hosts. An expression vector for use with microbes need only contain an origin of replication recognized by the host, a promoter which will function in the host, and a selection gene.

Commonly used promoters are derived from polyoma, bovine papilloma virus, CMV (cytomegalovirus, either murine or human), Rouse sarcoma virus, adenovirus, and simian virus 40 (SV40). Other control sequences (e.g., terminator, polyA, enhancer, or amplification sequences) can also be used.

An expression vector is constructed so that the chlorotoxin or chlorotoxin derivative polypeptide coding sequence is located in the vector with the appropriate regulatory sequences, the positioning and orientation of the coding sequence with respect to the control sequences being such that the coding sequence is transcribed and translated under the “control” of the control sequences (i.e., RNA polymerase which binds to the DNA molecule at the control sequences transcribes the coding sequence). The control sequences may be ligated to the coding sequence prior to insertion into a vector, such as the cloning vectors described above. Alternatively, the coding sequence can be cloned directly into an expression vector which already contains the control sequences and an appropriate restriction site. If the selected host cell is a mammalian cell, the control sequences can be heterologous or homologous to the chlorotoxin or chlorotoxin derivative polypeptide coding sequence, and the coding sequence can either be genomic DNA containing introns or cDNA.

Higher eukaryotic cell cultures may be used to express the proteins of the present invention, whether from vertebrate or invertebrate cells, including insects, and the procedures of propagation thereof are known.

Other expression vectors are those for use in eukaryotic systems. An exemplary eukaryotic expression system is that employing vaccinia virus, which is well-known in the art (see, for example, WO 86/07593). Yeast expression vectors are known in the art (see, for example, U.S. Pat. Nos. 4,446,235 and 4,430,428). Another expression system is vector pHSI, which transforms Chinese hamster ovary cells (see WO 87/02062). Mammalian tissue may be cotransformed with DNA encoding a selectable marker such as dihydrofolate reductase (DHFR) or thymidine kinase and DNA encoding the chlorotoxin or chlorotoxin derivative polypeptide. If wild type DHFR gene is employed, it is preferable to select a host cell which is deficient in DHFR, thus permitting the use of the DHFR coding sequence as marker for successful transfection in hgt medium, which lacks hypoxanthine, glycine, and thymidine.

Depending on the expression system and host selected, chlorotoxin or chlorotoxin derivative polypeptide are produced by growing host cells transformed by an exogenous or heterologous DNA construct, such as an expression vector described above under conditions whereby the polypeptide is expressed. The chlorotoxin or chlorotoxin derivative polypeptide is then isolated from the host cells and purified. If the expression system secretes the protein or peptide into the growth media, the protein can be purified directly from cell-free media. The selection of the appropriate growth conditions and initial crude recovery methods are within the skill of the art.

Once a coding sequence for a chlorotoxin or chlorotoxin derivative polypeptide of the invention has been prepared or isolated, it can be cloned into any suitable vector and thereby maintained in a composition of cells which is substantially free of cells that do not contain any chlorotoxin coding sequence. As described above, numerous cloning vectors are known to those of skill in the art.

Chlorotoxin Peptide Mimetics

In another class of chlorotoxin derivatives, the present invention includes peptide mimetics that mimic the three-dimensional structure of chlorotoxin. Such peptide mimetics may have significant advantages over naturally occurring peptides including, for example, more economical production, greater chemical stability, enhanced pharmacological properties (half-life, absorption, potency, efficacy, etc.), altered specificity (e.g., a broad-spectrum of biological activities), reduced antigenicity and others.

In one form, mimetics are peptide-containing molecules that mimic elements of chlorotoxin peptide secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly produced in the pharmaceutical industry as non-peptide drugs with properties analogous to those of the template peptide. These types of non-peptide compounds are also referred to as peptide mimetics or peptidomimetics (Fauchere (1986) Adv. Drug Res. 15, 29-69; Veber & Freidinger (1985) Trends Neurosci. 8, 392-396; Evans et al. (1987) J. Med. Chem. 30, 1229-1239 which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage by methods known in the art. Labeling of peptide mimetics usually involves covalent attachment of one or more labels, directly or through a spacer (e.g., an amide group), to non-interfering positions on the peptide mimetic that are predicted by quantitative structure-activity data and molecular modeling. Such non-interfering positions generally are positions that do not form direct contacts with the macromolecules to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of a peptide to, for instance, a tumor cell. Approaches that can be used include the yeast two hybrid method (see Chien et al. (1991) Proc. Natl. Acad. Sci. USA 88, 9578-9582) and using the phage display method. The two hybrid method detects protein-protein interactions in yeast (Fields et al. (1989) Nature 340, 245-246). The phage display method detects the interaction between an immobilized protein and a protein that is expressed on the surface of phages such as lambda and M13 (Amberg et al. (1993) Strategies 6, 2-4; Hogrefe et al. (1993) Gene 128, 119-126). These methods allow positive and negative selection for peptide-protein interactions and the identification of the sequences that determine these interactions.

Pharmaceutical Compositions

Pharmaceutical compositions of the present invention can be administered via parenteral, subcutaneous, intravenous, intramuscular, intraperitoneal, intrathecal, intracranial or transdermal or buccal routes. For example, an agent may be administered locally to a tumor via microinfusion. Alternatively, or concurrently, administration may be by the oral route. The dosage administered will be dependent upon the age, health, and weight of the recipient, kind of concurrent treatment, if any, frequency of treatment, and the nature of the effect desired.

While individual needs vary, determination of optimal ranges of effective amounts of each component is within the skill of the art. Dosages of chlorotoxin and/or derivatives thereof of the present invention typically comprise about 1.0 ng/kg body weight to about 0.13 mg/kg body weight. In one embodiment, dosages of chlorotoxin and/or derivatives thereof comprise about 1.0 ng/kg body weight to about 0.1 mg/kg body weight. In a preferred embodiment, dosages for systemic administration comprise about 0.01 μg/kg body weight to about 0.1 mg/kg body weight. In another embodiment, the dosage of chlorotoxin and/or derivatives thereof comprises less than about 0.1 mg/kg body weight. More preferred dosages for systemic administration comprise about 0.1 μg/kg body weight to about 0.05 mg/kg body weight. In another preferred embodiment, the dosage of chlorotoxin and/or derivatives thereof comprises less than about 0.05 mg/kg body weight. The most preferred dosages for systemic administration comprise between about 1.0 μg/kg body weight to about 0.01 mg/kg body weight. In other embodiments, the amount of chlorotoxin and/or derivatives thereof administered is an amount effective to bring the concentration of chlorotoxin and/or derivatives thereof in the serum to a concentration of about 20.0, 10.0, 5.0, 2.50, 1.25, 0.625, 0.3125, 0.156, 0.078, 0.039, 0.020, 0.010, 0.005, 0.003, 0.0015, 0.0008, 0.0003 or 0.0001 nM. The preferred dosages for direct administration to a site via microinfusion comprise 1 ng/kg body weight to 1 mg/kg body weight.

In addition to chlorotoxin and/or derivatives thereof, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

As mentioned above for some methods of the invention, topical administration may be used. Any common topical formulation such as a solution, suspension, gel, ointment or salve and the like may be employed. Preparation of such topical formulations are described in the art of pharmaceutical formulations as exemplified, for example, by Gennaro et al. (1995) Remington's Pharmaceutical Sciences, Mack Publishing. For topical application, the compositions could also be administered as a powder or spray, particularly in aerosol form. In a some embodiments, the compositions of this invention may be administered by inhalation. For inhalation therapy the active ingredients may be in a solution useful for administration by metered dose inhalers or in a form suitable for a dry powder inhaler. In another embodiment, the compositions are suitable for administration by bronchial lavage.

Suitable formulations for oral administration include hard or soft gelatin capsules, pills, tablets, including coated tablets, elixirs, suspensions, syrups or inhalations and controlled release forms thereof.

The invention also includes isotopically-labeled chlorotoxin derivatives that have one or more atoms replaced by an atom having an atomic mass or mass number different from the atomic mass or mass number usually found in nature, or one or more of such atoms attached to the chlorotoxin derivatives. Examples of isotopes that can be incorporated into compounds of the invention include, but are not limited to, isotopes of hydrogen, carbon, phosphorous, iodine, rhenium, indium, yttrium, technetium and lutetium (i.e., including, but not limited to, ³H, ¹⁴C, ³¹P, ³²P, ³⁵S, ¹³¹I, ¹²⁵I, ¹²³I, ¹⁸⁷Re, ⁶⁴Cu, ¹¹¹In, ⁹⁰Y, ^{99m}Tc, ¹⁷⁷Lu), others isotopes of these elements, and other isotopes known in the art. Agents of the present invention, prodrugs thereof, and pharmaceutically acceptable salts of said agents or of said prodrugs which contain the aforementioned isotopes and/or other isotopes of other atoms are within the scope of this invention. Tritium and carbon-14 isotopes are particularly preferred for their ease of preparation and detectability. Further, substitution with heavier isotopes such as deuterium can afford certain therapeutic advantages resulting from greater metabolic stability, for example increased in vivo half-life or reduced dosage requirements and, hence, may be preferred in some circumstances.

Fusion Proteins

The present invention also includes compositions where a cytotoxic agent is linked to a chlorotoxin derivative. Examples of cytotoxic agents include, but are not limited to, gelonin, ricin, saponin, pseudonomas exotoxin, pokeweed antiviral protein, diphtheria toxin, complement proteins, or any other agent known in the art which is capable of killing a cell upon contact with that cell.

The invention includes fusion polypeptides and salts thereof, comprising at least one second polypeptide. In some embodiments, the second polypeptide includes a cancer cell-binding domain which specifically binds to an epitope expressed only on cells exhibiting abnormal growth (i.e. cancer cells). The term “cancer cell-binding domain” refers to an amino acid sequence capable of binding or otherwise specifically associating with a cell displaying abnormal growth (e.g., benign and malignant cancer cells). In some embodiments the cancer cell binding-domain is an antibody while in other embodiments it is a ligand which specifically binds to a receptor expressed only on cancer cells. Examples of antibodies include, but are not limited to, antibodies which specifically bind to B-cells or T-cells. Examples of receptor ligands include, but are not limited to, cytokines and growth factors including epidermal growth factor.

The second polypeptide can also include a stabilization domain which increases the in vitro and in vivo half-life of the fusion polypeptide. As used herein, the term “stabilization domain” refers to an amino acid sequence capable of extending the in vitro and in vivo half-life of chlorotoxin or a chlorotoxin derivative when compared to chlorotoxin alone. The stabilization domain can comprise human proteins (e.g., full length or truncated, soluble proteins from extracellular fragments, etc) such as human serum albumin, transferrin or other proteins which stabilize the in vivo or in vitro half-life of chlorotoxin or a chlorotoxin derivative. These additional functional domains may themselves serve as linker peptides, for example, for joining a cancer cell-binding domain to chlorotoxin or a chlorotoxin derivative. Alternatively, they may be located elsewhere in the fusion molecule (e.g., at the amino or carboxy terminus thereof). In alternative embodiments, the stabilization domain is a chemical moiety (e.g., PEG (polyethylene glycol) or a dextran).

The term “fused” or “fusion polypeptide” as used herein refers to polypeptides in which: (i) a given functional domain (i.e. a cancer cell-binding domain) is bound at its carboxy terminus by a covalent bond either to the amino terminus of another functional domain (i.e., an human serum albumin component) or to a linker peptide which itself is bound by a covalent bond to the amino terminus of chlorotoxin or a chlorotoxin derivative; or (ii) a given functional domain (i.e. a cancer cell-binding domain) is bound at its amino terminus by a covalent bond either to the carboxy terminus of another functional domain (i.e., an human serum albumin component) or to a linker peptide which itself is bound by a covalent bond to the carboxy terminus of chlorotoxin or a chlorotoxin derivative.

Similarly, “fused” when used in connection with the nucleic acid intermediates of the invention means that the 3′-[or 5′-] terminus of a nucleotide sequence encoding a first functional domain is bound to the respective 3′-[or 5′-] terminus of a nucleotide sequence encoding a second functional domain, either by a covalent bond or indirectly via a nucleotide linker which itself is covalently bound preferably at its termini to the first functional domain-encoding polynucleotide and optionally, a second functional domain-encoding nucleic acid.

Examples of fusion polypeptides of the invention may be represented by, but are not limited by, the following formulas:
R1-L-R2 (i)
R2-L-R1 (ii)
R1-L-R2-L-R1 (iii)
R1-L-R1-L-R2 (iv)
R2-L-R1-L-R1 (iv)
wherein R1 is the amino acid sequence of a cancer cell-binding domain, R2 is the amino acid sequence of a stabilizing domain (e.g., human serum albumin), each L is chlorotoxin or a chlorotoxin derivative which is bound by a covalent bond to a terminus of R1 and/or R2, whereby the above molecule fragments are read directionally (i.e., with the left side corresponding to the amino terminus and the right side to the carboxy terminus of the molecule).
Methods of Treatment Using Chlorotoxin and/or Derivative Thereof

This invention includes methods for the treatment of abnormal cell growth in a mammal, including a human, comprising administering to said mammal an amount of chlorotoxin and/or derivatives thereof, an amount of a fusion protein comprising chlorotoxin or a derivative thereof, or a pharmaceutical composition comprising an amount of chlorotoxin and/or derivatives thereof, that is effective in inhibiting or arresting the growth of abnormally proliferating cells, such as cancer cells, without the addition of other therapeutic agents. In one embodiment of this method, the abnormal cell growth is cancer, including, but not limited to, prostate cancer, breast cancer, lung cancer, non-small cell lung carcinoma, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma, pituitary adenoma, or a combination of one or more of the foregoing cancers. In another embodiment of said method, said abnormal cell growth is a benign proliferative disease, including, but not limited to, psoriasis, benign prostatic hyperplasia, hypertrophy or restinosis.

This invention also includes methods for the treatment of abnormal cell growth in a mammal which comprises administering to said mammal, including a human, a pharmaceutical composition comprising an amount of chlorotoxin and/or derivatives thereof that is effective in inhibiting abnormal cell growth. This includes the abnormal growth and/or proliferation of cancer cells including benign and malignant cells of neoplastic diseases. Inhibition of abnormal cell growth can occur by a variety of mechanism including, but not limited to, cell death, apoptosis, inhibition of cell division, transcription, translation, transduction, etc.

In practicing the methods of this invention, chlorotoxin and/or derivatives thereof may be used alone or in combination with other inactive ingredients. As discussed above, the present invention includes compositions and methods where a drug or cytotoxic agent is linked to a chlorotoxin derivative. The methods of the invention therefore include administration of a chlorotoxin derivative linked to a cytotoxic agent for the treatment of a disease associated with abnormal cell growth, including cancer. Examples of cytotoxic agents include, but are not limited to, gelonin, ricin, saponin, pseudonomas exotoxin, pokeweed antiviral protein, diphtheria toxin, complement proteins, or any other agent known in the art which is capable of killing a cell upon contact with that cell.

The compositions and methods of the invention can be utilized in vivo, ordinarily in mammals, such as humans, sheep, horses, cattle, pigs, dogs, cats, rats and mice or in vitro. The invention is particularly useful in the treatment of human subjects.

Without further description, it is believed that one of ordinary skill in the art can, using the preceding description and the following illustrative examples, make and utilize the compounds of the present invention and practice the claimed methods. The following working examples describe embodiments of the present invention, and are not to be construed as limiting in any way the remainder of the disclosure.

### EXAMPLE 1

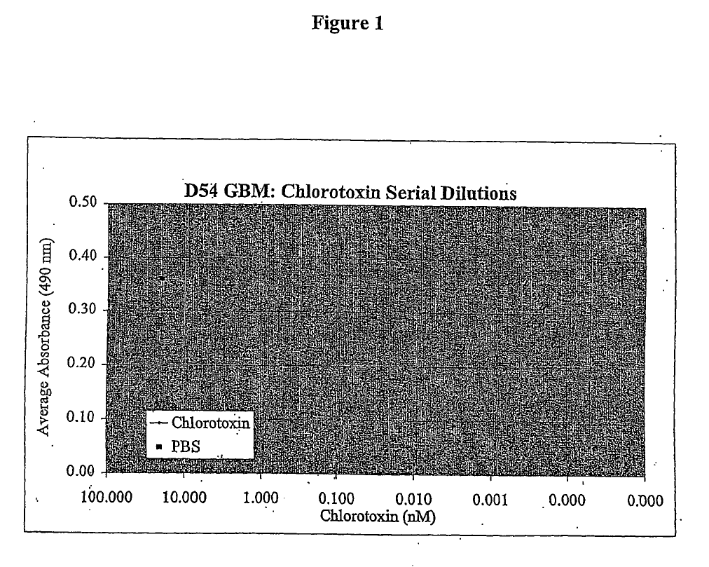

D54 glioblastoma cells were plated at a density of about 1000 cells/well in a 96-well flat bottom plate and incubated in 5% CO₂ at 37° C. After twenty-four hours chlorotoxin was added at 1:4 limiting dilutions to a final concentration of 20, 5, 1.25, 0.313, 0.078, 0.0195, 0.0049, 0.0012, 0.00031 or 0.00008 nM. Control cells received vehicle only. Twenty-four hours after treatment, the effect of chlorotoxin was quantified using the MIT mitochondrial enzyme substrate with the Cell Counting Kit-8 (CCK-8) (Dojindo Inc.) according to the manufacturer's instructions. In brief, following the treatment period with chlorotoxin, cells were incubated with CCK-8 reagent. After incubation, plates were read on a microplate reader at a wavelength of 490 nm, with higher absorbance indicating greater cell viability. FIG. 1 shows that chlorotoxin incubation inhibited proliferation of the D54 cells at all concentrations tested down through 0.00120 nM as evidenced by the lower number of viable cells/well versus PBS control.

### EXAMPLE 2

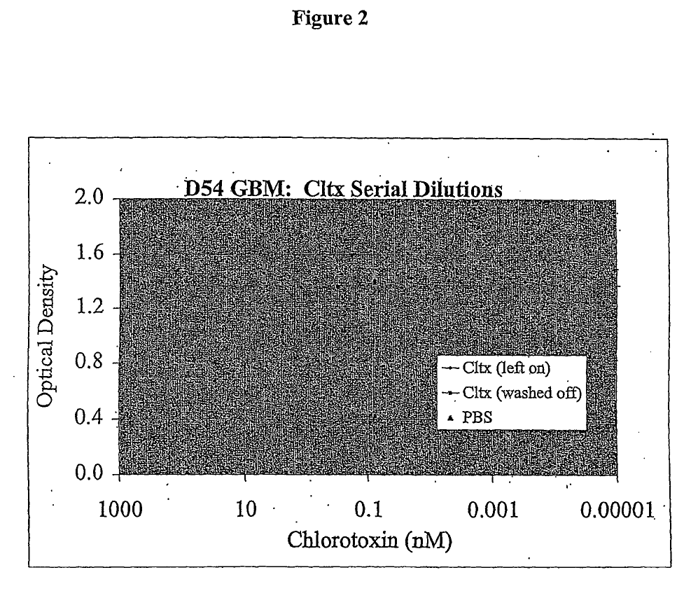

D54 glioblastoma cells were plated at a density of about 1000 cells/well in a 96-well flat bottom plate and incubated in 5% CO₂ at 37° C. After twenty-four hours chlorotoxin was added at 1:4 limiting dilutions to a final concentration (in nM) of 20, 5, 1.25, 0.313, 0.078, 0.02, 0.0049, 0.0012, 0.0003, or 0.00008. Control cells received vehicle only. After twenty-four hours, half of the cells were washed free of chlorotoxin, the medium replaced with fresh medium. Cells in both conditions, chlorotoxin left on and chlorotoxin removed, were incubated for an additional four days. Following incubation, the effect of chlorotoxin was quantified using the MTT mitochondrial enzyme substrate with the CCK-8 as in Example 1. FIG. 2 shows that the long incubation time allowed the cells to overcome the effects of chlorotoxin with the additional days of proliferation and chlorotoxin did not appear to inhibit cell proliferation in this instance.

### EXAMPLE 3

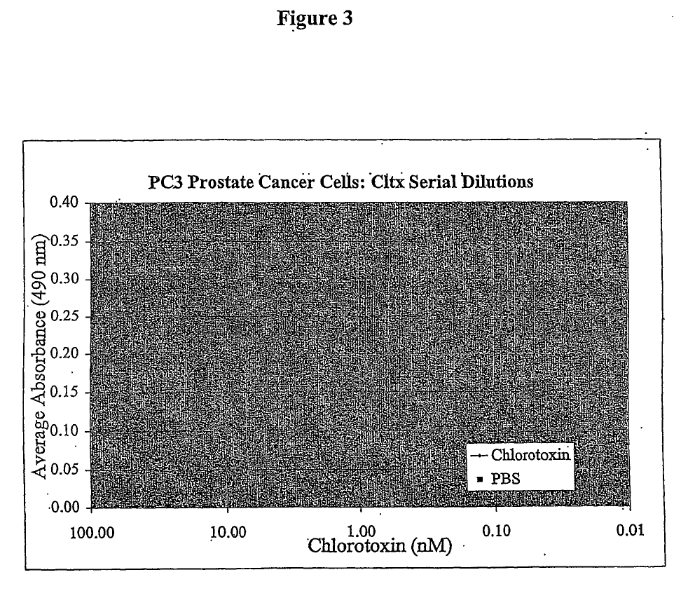

PC3 prostate cancer cells were plated at a density of about 1000 cells/well in a 96-well flat bottom plate and incubated in 5% CO₂ at 37° C. After twenty-four hours chlorotoxin was added at 1:2 limiting dilutions to a final concentration (nM) of 20, 10, 5, 2.5, 1.25, 0.625, 0.313, 0.156, 0.078, and 0.039. Control cells received PBS vehicle only. Twenty-four hours after treatment, the effect of chlorotoxin was quantified using the MTT mitochondrial enzyme substrate with the CCK-8 as in Example 1. FIG. 3 shows that chlorotoxin incubation inhibited proliferation of the D54 cells at all concentrations tested as evidenced by the lower number of viable cells/well versus PBS control

### EXAMPLE 4

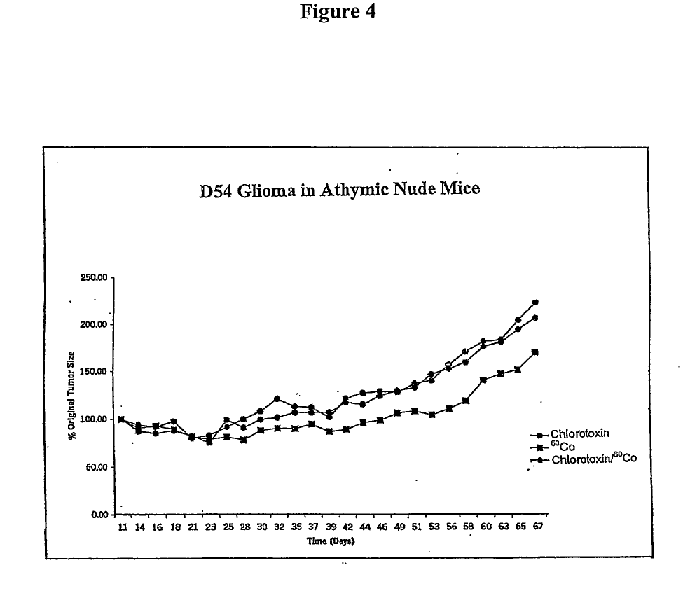

Three groups of eight athymic nude mice received a subcutaneous injection of 5×10⁷ human D54 glioblastoma cells in their right flank to produce human glioma flank xenografts in these mice. Animals in Groups I and III received 2.6 μg chlorotoxin (SEQ ID NO: 1) in 100 μl phosphate-buffered saline intravenously at 14, 21, 28, 35, 42 and 49 days after D54 injection. Animals in Groups II and III received 2 Gy ⁶⁰C whole-body irradiation at 15, 22, 29, 36, 43 and 50 days after D54 injection. Tumor size was measured three times weekly and is depicted in FIG. 4.

### EXAMPLE 5

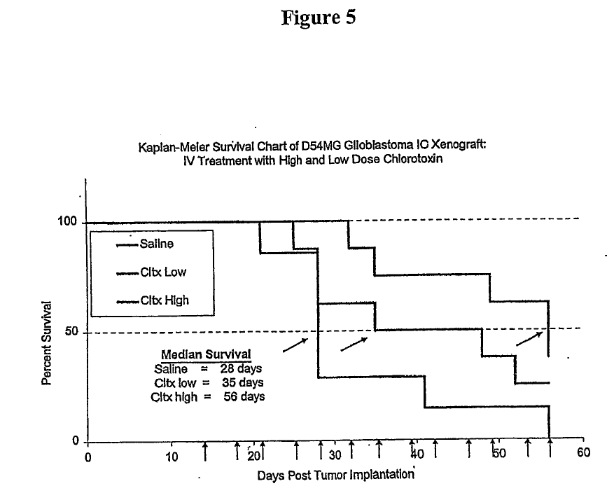

Intracranial D54MG glioma xenografts were established in athymic nude mice by the implantation of 1×10⁶ D54MG cells in the brain of each subject. A treatment regimen was begun 14 days post-implantation with tail vein intravenous injections two times per week. The control group of seven mice were administered saline vehicle only. A second group of mice, comprising eight animals, were each administered a low dose of chlorotoxin of 0.2 μg/dose and a third group of mice, comprising eight animals, were administered a high dose of chlorotoxin of 2.0 μg/dose. Animals were followed until death and survival time was plotted on a Kaplan-Meier chart, indicating median survival (FIG. 5). These results indicate that treatment with chlorotoxin alone substantially extends the life of a subject in an intracranial model and that this enhanced survival may be dose dependent. It is notable that administration of chlorotoxin was intravenous, demonstrating that chlorotoxin crosses the blood-brain barrier to exert its effect.

### EXAMPLE 6

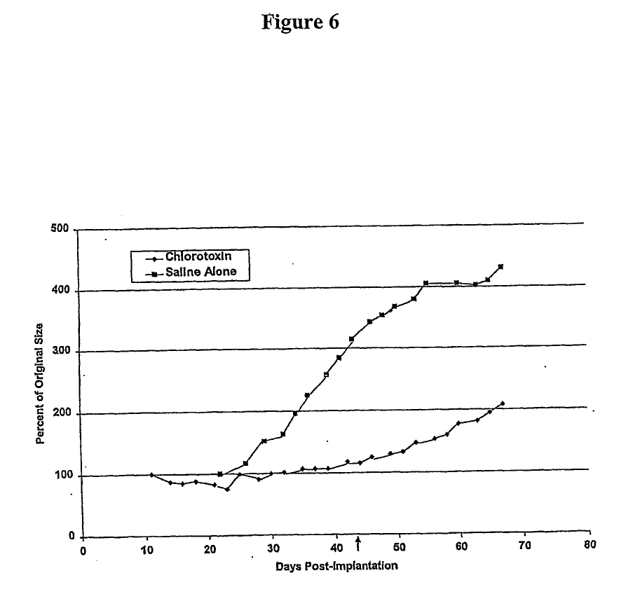

In a separate investigation, D54MG glioma xenografts were established peripherally by implanting 10×10⁶ D54MG cells in the flanks of athymic nude mice. Tumors were palpable at 14 days, with individual tumor volumes of approximately 43 mm³. Again, the treatment regimen was begun 14 days post-implantation with tail vein intravenous injections two times per week. The control group of seven mice were administered saline vehicle only. A second group of mice, comprising eight animals, were each administered a low dose of chlorotoxin of 0.2 μg/dose. Tumor size was measured at the time of each injection, and plotted as a percent of original tumor size (FIG. 6). Intravenous treatment was ended at 42 days and the measurement of the tumors was continued for several weeks. These results demonstrate that low-dose chlorotoxin alone can dramatically decrease the tumor growth in this flank model.

### EXAMPLE 7

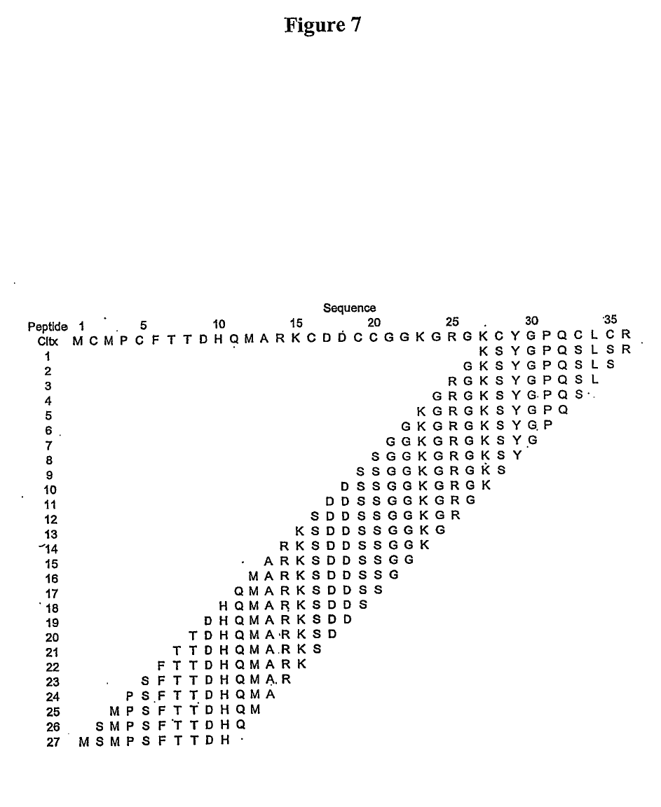

To identify core binding site sequences of chlorotoxin, twenty-seven overlapping 10-mers derived from SEQ 1) NO: 1 were synthesized, starting from the C-terminus of the peptide as indicated in FIG. 7. Each peptide had a biotin attached at the amino terminus to facilitate detection and each cysteine residue was replaced with a serine in order to prevent cross-linking.

Binding of the 10-mer peptides to PC3 prostate cancer cells in vitro was measured by incubating cultured PC3 cells with individual peptides. Binding was detected and quantified by incubating the peptide exposed cells with HRP-avidin using a commercial kit according to manufacturer's instructions.

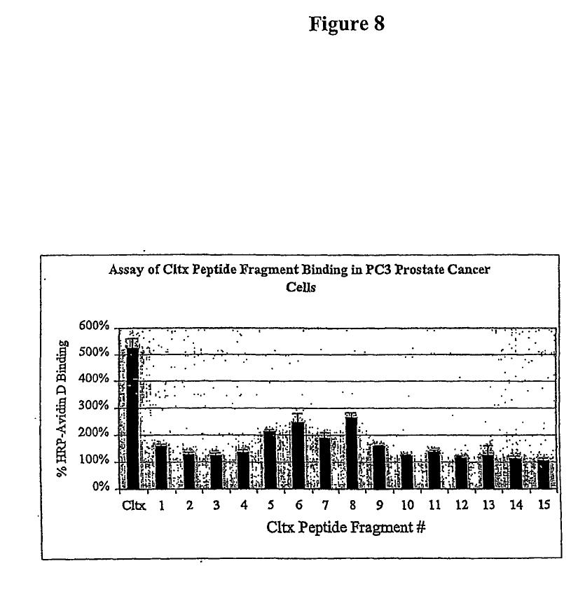

FIG. 8 shows that the 10-mer peptide 4 of SEQ ID NO: 1 does not bind to PC3, indicating that the lysine residue which starts peptide 5 must be the start of the binding site. Peptides 5-8 bind, but the binding is lost in peptide 9. This suggests that the tyrosine residue is another key, since this is present in peptide 8 but lost in peptide 9. This indicated that a first binding region of chlorotoxin resides within the 7-mer sequence KGRGKSY (SEQ ID NO: 8) residing at amino acid residues 23-29 of SEQ ID NO: 1 which are common to peptides 5-8.

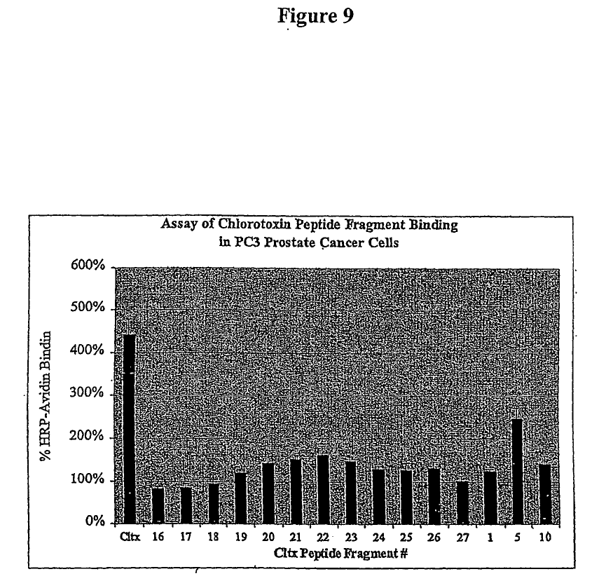

FIG. 9 shows that peptide 19 of SEQ ID NO: 1 does not bind PC3 cells but peptide 20 does, indicating that the threonine residue which starts peptide 20 may be the start of a second binding site because peptides 20-24 bind most strongly. Binding decreases again in peptide 25, suggesting that the peptide 24 terminal arginine residue is another key, since this is present in peptide 24 but lost in peptide 25. This indicates that a second binding region of chlorotoxin resides within the 9-mer sequence TDHQMAR (SEQ ID NO: 9) residing at amino acid residues 8-14 of SEQ ID NO: 1 which are common to peptides 20-24. The binding in this second core sequence is broader, which may be a reflection of very similar amino acids present at the ends of the region. For example, there are two threonine residues at peptides 20 and 21, and there is a lysine at the end of peptide 22 next to the arginine residue.

### EXAMPLE 8

To determine the in vivo activity of these identified binding regions, 10-mer peptides 5 (amino acid residues 23-32), 12 (amino acid residues 16-25; as a negative control) and 21 (amino acid residues 7-16) of SEQ ID NO: 1 were used in a crayfish paralysis assay, an assay which is commonly used to determine the bio-activity of chlorotoxin (see DeBin et al. (1993) Am. J. Physiol. 264, C361-369). Peptides 5 and 12 failed to paralyze crayfish, while peptide 21 was effective, indicating that the site which is responsible for the paralytic effect of chlorotoxin is the region defined by peptide 21.

Additionally, several of the chlorotoxin derivatives were each analyzed in the crayfish assay and compared to chlorotoxin (Table 2). Each of these derivatives comprises the putative end-amino acids, the T and the R within the sequence corresponding to peptide 21.

TABLE 2

| | | | | |
|---|---|---|---|---|
| | | Cray- | | |
| | SEQ | fish | | |
| Peptide | ID | Assay | Identity | Sequence Comparison |

| | | | | | |
|---|---|---|---|---|---|
| Cltx | 1 | Yes | 100% | TDHQMAR | (SEQ ID NO: 9) |
| Cltx | 5 | Yes | 100% | TDHQMAR | (SEQ ID NO: 9) |
| (Y/F) | | | | | |
| STP-1 | 6 | Yes | 71.4% | TDPQMSR | (SEQ ID NO: 77) |
| 6xH-Cltx | 2 | Yes | 100% | TDHQMAR | (SEQ ID NO: 9) |
| Y-Cltx | 3 | Yes | 100% | TDHQMAR | (SEQ ID NO: 9) |
| YSY-Cltx | 4 | Yes | 100% | TDHQMAR | (SEQ ID NO: 9) |

### EXAMPLE 9

Chlorotoxin is a 36-amino acid peptide with 8 cysteines, depicted below in bold type with the sequences of peptide number 8 (beta-region peptide) and peptide number 21 (alpha-region peptide) identified using the overlapping 10-mers in Example 8 underlined:

| | |
|---|---|
| MCMPCFTTDHQMARKCDDCCGGKGRCKCYGPQCLCR | (SEQ ID NO: 1) |

In order to confirm the identify the minimal binding sequences within the alpha and beta peptides, the entire peptides were synthesized as a 10-mer with a biotin at the amino terminus as well as shorter sequences reducing the size of the peptide by one amino acid at the amino terminus each time.

For the beta peptide, the sequences of peptide 8 noted in Table 3 were evaluated and probed for binding to U251 glioma cells:

TABLE 3

| | |
|---|---|
| Peptide | Sequence |

| | | |
|---|---|---|
| 8 | Biotin-GGKGRGKSYG | (SEQ ID NO: 78) |
| 8a | Biotin-GKGRGKSYG | (SEQ ID NO: 79) |
| 8b | Biotin-KGRGKSYG | (SEQ ID NO: 80) |
| 8c | Biotin-GRGKSYG | (SEQ ID NO: 81) |

For the alpha peptide, the sequences of peptide 21 noted in Table 4 were evaluated and probed for binding to U251 glioma cells:

TABLE 4

| | |
|---|---|
| Peptide | Sequence |

| | | |
|---|---|---|
| 21 | Biotin-TTDHQMARKS | (SEQ ID NO: 82) |
| 21a | Biotin-TDHQMARKS | (SEQ ID NO: 10) |
| 21b | Biotin-DHQMARKS | (SEQ ID NO: 83) |
| 21c | Biotin-HQMARKS | (SEQ ID NO: 84) |
| 21d | Biotin-QMARKS | (SEQ ID NO: 85) |

Results demonstrated that the initial threonine residue of the alpha-region peptide is detrimental to binding but that the second threonine is crucial to binding. It was also discovered that none of the smaller peptides exhibit binding as strong as the 9-mer of peptide 21a.

### EXAMPLE 10

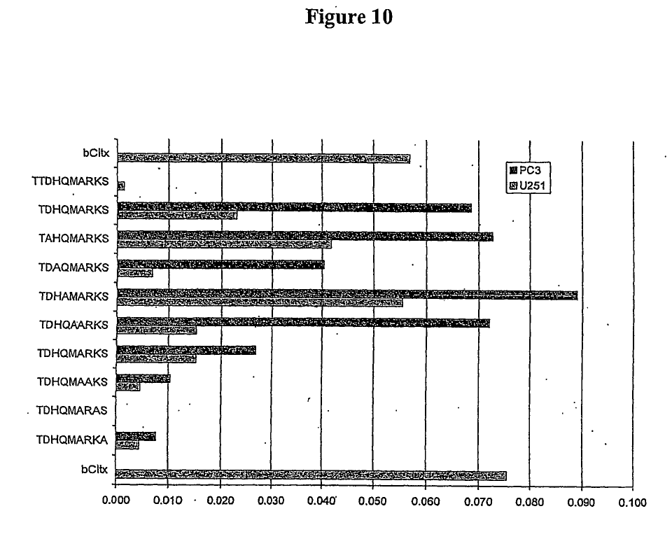

To determine the contribution of each residue to the binding properties of the alpha peptide, alanine scan variants were synthesized by replacing each amino acid of the 9-mer peptide TDHQMARKS (SEQ ID NO: 10) sequentially as depicted in Table 5. Peptide 21, the native core 9-mer, and each alanine-substituted 9-mer peptide was synthesized with a biotin at the amino terminus and evaluated for their binding versus both U251 and PC3 cells (FIG. 10).

TABLE 5

| | |
|---|---|
| Peptide | Sequence |

| | | |
|---|---|---|
| 21 | Biotin-TTDHQMARKS | (SEQ ID NO: 82) |
| 21a | Biotin-TDHQMARKS | (SEQ ID NO: 10) |
| 21a-A1 | Biotin-ADHQMARKS | (SEQ ID NO: 86) |
| 21a-A2 | Biotin-TAHQMARKS | (SEQ ID NO: 87) |
| 21a-A3 | Biotin-TDAQMARKS | (SEQ ID NO: 88) |
| 21a-A4 | Biotin-TDHAMARKS | (SEQ ID NO: 89) |
| 21a-A5 | Biotin-TDHQAARKS | (SEQ ID NO: 90) |
| 21a-A6 | Biotin-TDHQMARKS | (SEQ ID NO: 10) |
| 21a-A7 | Biotin-TDHQMAAKS | (SEQ ID NO: 91) |
| 21a-A8 | Biotin-TDHQMARAS | (SEQ ID NO: 92) |
| 21a-A9 | Biotin-TDHQMARKA | (SEQ ID NO: 93) |

The pattern for U251 and PC3 binding are generally similar. Replacement of the aspartic acid (D) residue in the second position of the 9-mer increased binding of the peptide to cells and replacement of the Q residue in the fourth position produced a large increase of peptide binding to cells. Accordingly, the peptide TAHAMARKS (SEQ ID NO: 11) should be more active than the parent peptide TDHQMARKS (SEQ ID NO: 10). Based on the binding of the peptide TDHAMARKS, this binding may be equal to or greater than chlorotoxin itself.

Based upon this finding, it is expected that a variant peptide of chlorotoxin of the sequence below may be stronger in binding than the native chlorotoxin polypeptide.

(SEQ ID NO: 12)

MCMPCFTTAHAMARKCDDCCGGKGRCKCYGPQCLCR

### EXAMPLE 11

In order to compare binding of the short scorpion toxins, the regions homologous to peptide 21 of small toxin and probable toxin LQH-8/6 were synthesized and biotinylated for analysis in the chlorotoxin binding assay. (See Table 6 for amino acid sequences of the peptides).

TABLE 6

| | |
|---|---|
| Scorpion Toxin | Peptide 21 |

| | | |
|---|---|---|
| Chlorotoxin | TTDHQMARKS | (SEQ ID NO: 82) |
| Small Toxin | TTDPQMSKK | (SEQ ID NO: 94) |
| Probable Toxin LQH-8/6 | TTDQQMTKK | (SEQ ID NO: 95) |

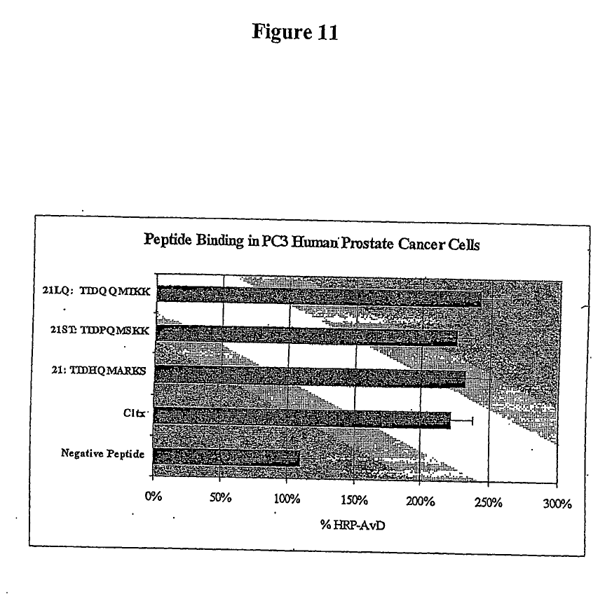

As shown in FIG. 11, and in accordance with previous results, chlorotoxin exhibited significant binding in PC3 human prostate cancer cells (221.93% of background levels) and peptide 21 binding paralleled that of chlorotoxin (232.50% of background levels). Additionally, peptide 21 of small toxin peptide (21ST) and peptide 21 of probable toxin LQH-8/6 (21LQ) demonstrated binding levels equivalent to that of full-length chlorotoxin and chlorotoxin peptide 21 (225.26% and 242.32%, respectively). Furthermore, a negative peptide containing amino acids 26-35 of chlorotoxin (SEQ ID NO: 1) exhibited binding levels comparable to background (110%). Similar results were obtained in D54 glioblastoma cells (data not shown).

The results from this study using the chlorotoxin binding assay indicate that chlorotoxin, small toxin peptide, and probable toxin LQH-8/6 bind similarly to human cancer cells in vitro. Table 7 below highlights amino acids conserved within the putative primary binding domain (amino acids 7-16) of the three toxin peptides.

TABLE 7

| | |
|---|---|
| Scorpion toxin | Amino acid sequence |

| | | |
|---|---|---|
| Chlorotoxin | TTDHQMARKC | (SEQ ID NO: 61) |
| Small toxin peptide | TTDPQMSKK | (SEQ ID NO: 94) |
| Probable toxin LQH-8/6 | TTDQQMTKK | (SEQ ID NO: 95) |

### EXAMPLE 12

The purpose of this experiment was to determine if the proliferation D54MG Glioblastoma cells, as measured by ³H-thymidine uptake, is effected by Peptide 21, a segment of the full chlorotoxin sequence. The sequence of peptide 21 and its relation to chlorotoxin is shown in the sequence below:

| | |
|---|---|
| Chlorotoxin: | MCMPCFTTDHQMARKCDDCCGGKGRGKCYGPQCLCR |
| Peptide 21: | TTDHQMARK (SEQ ID NO: 82) |

Peptide 21 (SEQ ID NO: 82) has been identified in several other reports as having binding and biological activity comparable to the fall length chlorotoxin.

D54MG cells were plated in a 24 well plate at 100,000 cells/ml/well using five rows of four wells for each concentration. The cells were allowed to adhere in normal media for twenty-four hours at 37° C. and 5% carbon dioxide. TM-701 was diluted to a 1 nM stock solution and added to each row at the concentrations of 0, 20, 80, 160 and 320 nM.

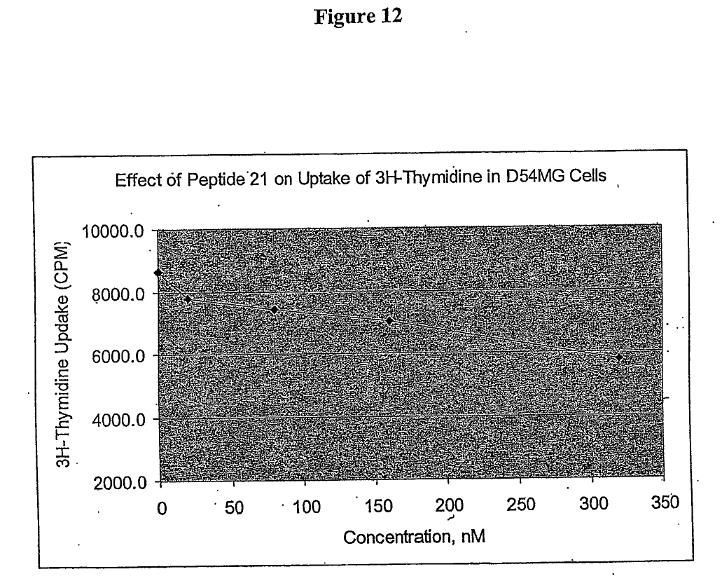

The cells and peptide 21 were allowed to incubate for 24 hours at 37° C. and 5% carbon dioxide. After twenty-four hours, the cells were rinsed two times with warm PBS. Normal media was added back to the cells at 1 ml/well. One μCi of ³H-thymidine was added to each well (1 μl of 1 mCi/ml ³H-thymidine to each well). The plate was incubated for two hours at 37° C. The media and thymidine were removed and the wells were rinsed with ice-cold phosphate-buffered saline three times. To each well was added 1 ml of 0.3 N NaOH. The plate was incubated in the 37° C. incubator for thirty minutes. Each well of 0.3 N NaOH was pipetted up and down three to four times and removed from the plate and the solution was placed in scintillation vials for counting. Scintillation fluid at four times the amount of sample was added to the vials (4 ml). Each vial was counted on the scintillation counter for one minute. The results are shown in Table 8 and FIG. 12. The data demonstrates that peptide 21 behaves similar to chlorotoxin, in that the uptake of ³H-thymidine decreases in a does-dependent manner. This data also indicates that peptide 21 has an effect on the DNA synthesis in these cells.

TABLE 8

| [Peptide 21] (nM) | ³H-Thymidine uptake ± SD (CPM) | |
|---|---|---|
| 0 | 8645 ± 1218 | 1218 |
| 20 | 7795 ± 634 | 634 |
| 80 | 7412 ± 630 | 630 |
| 160 | 6983 ± 329 | 329 |
| 320 | 5782 ± 886 | 886 |

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1. An isolated polypeptide comprising a subunit of chlorotoxin or a related scorpion toxin, wherein the polypeptide specifically binds to a cancer cell.

2. The isolated polypeptide of claim 1 wherein the polypeptide comprises an amino acid sequence selected from the group consisting of SEQ ID NO: 6, 7, 8, 9, 10, 11, 12, 13 and 14.

3. The isolated polypeptide of claim 1 wherein the polypeptide comprises the amino acid sequence
TTX₁X₂X₃MX₄X₅K (SEQ ID NO: 13), wherein
(a) X₁ is an acidic amino acid selected from the group consisting of aspartic acid and glutamic acid;
(b) X₂ is an amino acid selected from the group consisting of alanine, arginine, asparagine, aspartic acid, cysteine, glutamine, glutamic acid, glycine, histidine, isoleucine, leucine, lysine, proline, methionine, phenylalanine, serine, threonine, tryptophan, tyrosine and valine;
(c) X₃ is an amide amino acid selected from the group consisting of asparagine and glutamine;
(d) X₄ is an amino acid selected from the group consisting of serine, threonine and alanine; and
(e) X₅ is a basic amino acid selected from the group consisting of histine, lysine and arginine.

4. The isolated polypeptide of claim 3 wherein the amino acid sequence is SEQ ID NO: 14 (TTDHQMARK).

5. The isolated polypeptide of claim 3 wherein the amino acid sequence is SEQ ID NO: 94 (TTDQQMTKK).

6. The isolated polypeptide of claim 3 wherein in the amino acid sequence is SEQ ID NO: 95 (TTDPQMSKK).

7. The isolated polypeptide of claim 1 wherein the cancer cell is from a cancer selected from the group consisting of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma.

8. A fusion polypeptide comprising the polypeptide of claim 1 linked to a second polypeptide.

9. The fusion polypeptide of claim 8 wherein the second polypeptide comprises a cancer cell-binding domain which binds specifically of an epitope expressed only by a cancer cell.

10. The fusion polypeptide of claim 9 wherein the second polypeptide is an antibody or fragment thereof.

11. The fusion polypeptide of claim 8 wherein the second polypeptide comprises a stabilization domain which prevent degradation of the fusion polypeptide.

12. The fusion polypeptide of claim 8 wherein the second polypeptide is selected from the group consisting of polyhistidine, human serum albumin and human transferrin.

13. The polypeptide of claim 1 wherein the polypeptide is linked to a cytotoxic agent.

14. The polypeptide of claim 13 wherein the cytotoxic agent is selected from the group consisting of gelonin, ricin, saponin, pseudonomas exotoxin, pokeweed antiviral protein, diphtheria toxin and complement proteins.

15. The polypeptide of claim 1 wherein the polypeptide is labeled.

16. The polypeptide of claim 15 wherein the label is ¹³¹I.

17. A composition comprising the polypeptide of claim 1.

18. A method of treating a disease characterized by abnormal cell proliferation in a mammal comprising administering the composition of claim 17.

19. The method of claim 18, wherein the disease is cancer.

20. The method of claim 19 wherein the cancer is selected from the group consisting of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma.

21. A method of treating a disease in a mammal characterized by abnormal cell proliferation comprising administering a composition consisting essentially of chlorotoxin or a related scorpion toxin.

22. The method of claim 21 wherein the composition is suitable for use in humans.

23. The method of claim 21 wherein the amount of chlorotoxin administered is less than about 0.1 mg/kg body weight.

24. The method of claim 21 wherein the amount of chlorotoxin administered is less than about 0.05 mg/kg body weight.

25. The method of claim 21 wherein the amount of chlorotoxin administered comprises between about 0.01 μg/kg body weight to about 0.1 mg/kg body weight.

26. The method of claim 21 wherein the amount of chlorotoxin administered comprises between about 0.1 μg/kg body Weight to about 0.05 mg/kg body weight.

27. The method of claim 21 wherein the amount of chlorotoxin administered comprises between about 0.1 μg/kg body weight to about 0.1 mg/kg body weight.

28. The method of claim 21 wherein the amount of chlorotoxin administered comprises between about 0.1 μg/kg body weight to about 1.0 mg/kg body weight.

29. The method of claim 21 wherein the amount of chlorotoxin administered comprises between about 0.1 μg/kg body weight to about 2.0 mg/kg body weight.

30. The method of claim 21 wherein the mammal is a human.

31. The method of claim 21 wherein the disease is cancer.

32. The method of claim 31 wherein the cancer is selected from the group consisting of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma.

33. An isolated nucleic acid molecule encoding the polypeptide of claim 1.

34. The isolated nucleic acid molecule of claim 33 wherein the nucleic acid molecule is operably linked to one or more expression control elements.

35. A vector comprising an isolated nucleic acid molecule of claim 34.

36. A host cell comprising the vector of claim 35.

37. The host cell of claim 36 wherein the host is selected from the group consisting of prokaryotic host cells and eukaryotic host cells.

38. A method for producing a polypeptide comprising culturing the host cell of claim 37 under conditions in which the polypeptide encoded by said nucleic acid molecule is expressed.

39. The method of claim 38 wherein the host cell is selected from the group consisting of prokaryotic host cells and eukaryotic host cells.

40. An isolated polypeptide produced by the method of claim 39.

41. A method for producing a polypeptide comprising chemically synthesizing the polypeptide of claim 1.
